(19) United States
(12) Patent Application Publication
(10) Pub. No.: US 2005/0276836 A1
(43) Pub. Date: Dec. 15, 2005

(54) Coated vaginal devices for vaginal delivery of therapeutically effective and/or health-promoting agents

(76) Inventors: Michelle Wilson, Hamilton, OH (US); Kishorkumar J. Desai, Westchester, OH (US); Giovanni M. Pauletti, Loveland, OH (US); Mitchell K. Antoon JR., Cincinnati, OH (US); Chris E. Clendening, Cleves, OH (US)

Correspondence Address: PETERS VERNY JONES & SCHMITT, L.L.P., 425 SHERMAN AVENUE, SUITE 230, PALO ALTO, CA, 94306, US

(21) Appl. No.: 11/180,076

(22) Filed: Jul. 12, 2005

Related U.S. Application Data
(63) Continuation-in-part of application No. 11/126,863, filed on May 10, 2005.
Application No. 11/126,863 is a continuation-in-part of application No. 10/226,667, filed on Aug. 21, 2002.
Application No. 11/126,863 is a continuation-in-part of application No. 10/600,849, filed on Jun. 20, 2003.
Application No. 11/126,863 is a continuation-in-part of application No. 10/349,029, filed on Jan. 22, 2003, now Pat. No. 6,905,701.
Application No. 10/349,029 is a continuation-in-part of application No. 09/626,025, filed on Jul. 27, 2000, now Pat. No. 6,572,874.
Application No. 09/626,025 is a continuation-in-part of application No. 09/249,963, filed on Feb. 12, 1999, now Pat. No. 6,086,909.
Application No. 09/249,963 is a continuation-in-part of application No. 09/079,897, filed on May 15, 1998, now Pat. No. 6,197,327.
(60) Provisional application No. 60/587,454, filed on Jul. 12, 2004.
(60) Provisional application No. 60/049,325, filed on Jun. 11, 1997.

(51) Int. Cl. A61F 13/00

(52) U.S. Cl. 424/422

(57) ABSTRACT

A vaginal device for delivering therapeutical and/or health-promoting agents. The vaginal device partly or completely coated by, covered by or combined with a coating or covering comprising a film, foam, strip, cap, cup or particles. The coating of the device comprises a mucoadhesive composition comprising a therapeutical and/or health-promoting agent.

This application claims priority of the Provisional application Ser. No. 60/587,454 and is a continuation-in-part of application Ser. No. 11/126,863 filed on May 10, 2005 and is a continuation-in-part of application Ser. No. 10/226,667 filed on Aug. 21, 2002 and a continuation-in-part of application Ser. No. 10/600,849, filed Jun. 20, 2003 and a continuation-in-part of application Ser. No. 10/349,029, filed Jan. 22, 2003, issued as U.S. Pat. No. 6,905,701, which is a continuation-in-part of application Ser. No. 09/626,025, filed Jul. 27, 2000, issued as U.S. Pat. No. 6,572,874 which is a continuation-in-part of application Ser. No. 09/249,963, filed Feb. 12, 1999, issued as U.S. Pat. No. 6,086,909, which is a continuation-in-part of the U.S. patent application Ser. No. 09/079,897, filed on May 15, 1998, issued as U.S. Pat. No. 6,197,327, which claims priority of the Provisional Application Ser. No. 60/049,325, filed Jun. 11, 1997, under 35 U.S.C. §111(b).

### BACKGROUND OF THE INVENTION

### FIELD OF THE INVENTION

The present invention concerns a vaginal device for delivering therapeutical and/or health-promoting agents. The device is either partly or completely coated by, covered by or combined with a coating or covering comprising a layer, film, foam, fiber, strip, suppository, pellet, tablet, soft gel capsule, cap, cup or particles. The coating form a one layer or several layers and may be dissolving or non-dissolving material. The coating of the device further comprises or is incorporated with a mucoadhesive composition comprising a therapeutical and/or health-promoting agent or a combination thereof suitable for delivery of said agent to the uterus and/or to the general circulation through vaginal mucosa. In particular, the invention concerns a targeted delivery of the therapeutical and/or health-promoting agents to the uterus or to the general circulation using the vaginal device such as a tampon, tampon-like device, vaginal foam, vaginal pessary, vaginal pellet, vaginal ring, vaginal suppository, vaginal pellet or vaginal patch covered with the one or several layers of the coating or covering, or is in combination with such covering, said coating or covering is formed of particles, foam, film or strip or is forming a cap, cup, film, foam, suppository, pellet, soft gel capsule or strip for attachment to or for combination with the vaginal device. The device allows delivery of the drug directly to the uterus or to the general systemic circulation in lower concentrations than those needed for systemic treatment and thus provides for lower systemic concentration and fewer side effects.

### BACKGROUND OF THE INVENTION

Systemic administration of therapeutical or palliative drugs, medicaments and natural products by the oral route to the patient has generally not been successful in relieving the condition in many women as the oral administration is frequently limited by side effects. This failure is believed to be the result of a failure to deliver and achieve an effective dosage level of these agents to the target site.

The vaginal delivery route of drugs through the vaginal mucosa to the uterus and to the general circulation has been discovered by inventors and is disclosed, for example, in the U.S. patent application Ser. No.09/079,897, filed on May 15, 1998 which claims priority from the copending, commonly assigned provisional application Ser. No. 60/049,325, filed Jun. 11, 1997, under 35 U.S.C. §111(b). U.S. Pat. No. 6,086,909 issued from the above applications.

As well as the vaginal delivery route described in the above cited patent works, there is some need for improvement. The current application concerns an improved transmucosal delivery through vaginal mucosa which is more efficacious due to a more quantifyable sequestration of the drug within the coating or covering of the vaginal device with one or several layers of coating material or covering said device completely or partially with such coating or covering or combining said vaginal device with such covering.

It is therefore a primary objective of this invention to provide a vaginal device coated, covered or combined with such coating or covering, with one or several layers of the coating, or covering said device with a film, foam, strip, cap or cup comprising a mucoadhesive composition containing and able to release a therapeutical or health-promoting agent therefrom and deliver it through the vaginal mucosa into the uterus and/or to the general circulation.

### SUMMARY OF THE INVENTION

One aspect of the current invention is a vaginal device for delivering a therapeutical or health-enhancing agent to a female subject wherein said vaginal device is a vaginal tampon, vaginal tampon-like device, vaginal ring, vaginal pessary, vaginal foam, vaginal suppository, vaginal tablet, vaginal pellet or vaginal patch and wherein said vaginal device is partly or completely coated by, covered by, or combined with, a covering comprising a film, strip, layer, cap, cup, foam, suppository, pellet, tablet, soft gel capsule or particles, said coating or covering further comprising the therapeutical or health-enhancing agent.

Another aspect of the current invention is a vaginal device coated, covered or combined with a covering comprising a therapeutical or health-enhancing agent formulated as a mucoadhesive composition incorporated into said coating or covering of said device.

Still another aspect of the current invention is the vaginal device coated or covered with a coating comprising a mucoadhesive composition containing at least one therapeutical agent selected from the group consisting of an antimicrobial, vasodilator, nonsteroidal anti-inflammatory (NSAI), prostaglandin inhibitor, COX-1 inhibitor, COX-2 inhibitor, local anesthetic, calcium channel antagonist, potassium channel blocker, β-adrenergic agonist, bisphosphonate, leukotriene blocker, smooth muscle inhibitor, peptide, protein, dyskinetic muscle contraction inhibitor and anti-HIV agent and a combination thereof, or at least one health-promoting agent selected from the group consisting of a botanical, probiotic microorganism, vitamin, herb, mineral, enzyme, co-enzyme, co-factor, antioxidant, anti-pruritic additive, synergistic additive agent, extract from a natural product, terpenoid, alkaloid, shikimate, polyketide, and a combination thereof.

Still another aspect of the current invention is a vaginal device wherein a mucoadhesive composition comprising a therapeutical or health-promoting agent is incorporated into a coating or furnished as an attachable covering for said device.

Still yet another aspect of the current invention is a vaginal device coated with a film, foam, strip or particle coating or covered with a film, foam, strip, cup or cap covering wherein said coating or covering comprises a mucoadhesive composition comprising a suitable biocompatible excipient in combination with a therapeutical and/or health-promoting agent.

Yet another aspect of the current invention is a mucoadhesive composition comprising a therapeutical and/or health-promoting agent and further comprising an excipient selected from a lipophilic carrier such as semi-synthetic glyceride of saturated fatty acids, a hydrophilic carrier such as polyethylene glycol having an average molecular weight of 6000, polyethylene glycol having an average molecular weight of 1500, polyethylene glycol having an average molecular weight of 400 or mixtures thereof, a muco-adhesive agent such as alginate, pectin, or cellulose derivative, and a penetration enhancer such as bile salts, organic solvents, ethoxydiglycol or interesterified stone oil.

Still another aspect of the current invention is a mucoadhesive composition for incorporation into a coating or covering for a vaginal device said composition comprising an excipient present in amounts between about 60 to 90% by weight lipophilic or hydrophilic carrier, between about 5 to 25% mucoadhesive agent, and between about 5 to 20% penetration enhancer.

In another aspect, the invention provides a vaginal tampon or a tampon-like device for delivering a therapeutical and/or health-promoting agent to the uterus said device comprising a cup or cap-shaped portion further covered with a cup or cap porous foam, film or strip of material incorporated with a mucoadhesive composition comprising a therapeutical and/or health-promoting agent.

In yet another aspect of the current invention, the vaginal device coated or covered with a coating or covering comprises a mucoadhesive composition formulated as a bioadhesive microparticle, cream, tablet, soft gel capsule, lotion, foam, ointment, solution or gel, all in regular, controlled or sustained controlled release form.

### BRIEF DESCRIPTION OF THE DRAWINGS

FIG. 1A is a graph illustrating pharmacokinetic of an anti-migraine drug sumatriptan in female white New Zealand rabbits following vaginal administration. Each animal received 0.7 mg of sumatriptan per kg body weight. Open circles (◯) denote plasma concentration following intravenous injection, closed squares (▪) represent corresponding to the drug plasma concentration after oral dosing. Results are presented as means±S.D. FIG. 1B shows systemic plasma concentrations of anti-migraine drug sumatriptan in female white New Zealand rabbits after vaginal insertion of a lipophilic vaginal delivery device. Each animal received 0.7 mg of sumatriptan per kg body weight and plasma concentrations were normalized to cₘₐₓ measured in similar experiments after oral administration of a dose-equivalent sumatriptan solution. Studies were performed in three different animals and results were presented as means±S.D.

FIG. 2A shows an anti-nausea agent metoclopramide plasma concentrations in female white New Zealand rabbits after intravenous administration. Drug solution comprising 0.5 mg of metoclopramide was prepared in sterile saline (0.9%; w/w) and injected into the systemic circulation through the marginal ear vein. Plasma samples were collected for six hours and analyzed for the drug using HPLC. Results are presented as means plasma±SEM (n=4). FIG. 2B shows normalized metoclopramide plasma concentrations in white female New Zealand rabbits after vaginal administration. Plasma samples were analyzed for the drug using a selective HPLC method and normalized to metoclopramide concentrations measured at the same time points as those used for intravenous injection. Experiments were performed in three individual animals and results are presented as mean±SEM.

FIG. 3 is a graph showing time release (%) of ketorolac tromethamine from sodium alginate/hydroxypropyl methyl cellulose foams in phosphate buffer (pH 4.22) at ketorolac concentration 7.4%, normalized to 120 mg of foam.

FIG. 4 is a graph showing time release (%) of ketorolac tromethamine from two-layer suppositories having an active and inactive layer.

FIG. 5 is a graph showing ketoconazole release (μg/ml) from 50:50 and 25:75 alginic acid/hydroxy propyl methylcellulose with 20 mg ketoconazole in phosphate buffer (pH 7.0).

FIG. 6 is a cross-sectional representation of a portion of the female reproductive organs including the uterus and vagina in the upright orientation.

FIG. 7 is a cross-sectional side view representation of a portion of the female reproductive organs including the uterus and vagina.

FIG. 8 shows placement of a vaginal tampon or a tampon-like foam device covered with a cap comprising a mucoadhesive composition containing an anti-migraine or anti-nausea drug incorporated therein or attached thereto.

FIG. 9 is a cross-sectional side view representation of the vaginal area adjacent the cervix showing placement of one embodiment of a vaginal tampon or a tampon-like foam coated with a layer or layers of a material incorporated with a mucoadhesive composition containing an anti-migraine or anti-nausea drug.

FIG. 10 shows placement of a vaginal tampon or a tampon-like foam device covered with a layer of material insulating the device from a material containing a mucoadhesive composition wherein the material forms a protective skirt-like around the proximal medicated portion of the device placed into a close proximity of the vaginal mucosa.

FIG. 11 shows an alternate embodiment of the tampon or a tampon-like foam device coated with a coating wherein a mucoadhesive composition is incorporated into the porous foam cap separated from the body of the tampon by the layer wherein the foam material used for the device is different from the foam device material used for formation of the foam cup.

FIG. 12 shows a tampon partially coated at its proximate end with a coating wherein the proximal end of the tampon or a tampon-like foam is incorporated with a mucoadhesive composition comprising an anti-migraine or anti-nausea drug, said composition formulated as a tablet, suppository or gel capsule.

FIG. 13 shows a tampon or a tampon-like foam device coated with a layer of material forming a cap with protruding fingers, said cap or only the fingers being incorporated with a mucoadhesive composition comprising an anti-migraine or anti-nausea drug.

FIG. 14 shows a partially coated tampon or a tampon-like foam device at its proximal end with a layer of material forming a cap incorporating a scoop-shaped gel capsule comprising a mucoadhesive composition of the invention.

FIG. 15 shows a tampon device covered with a cap, strip or fiber covering where the cap is positioned over the proximal end of the vaginal tampon (FIG. 15A) or where the coating or covering cap is positioned over the inserting tube of the tampon applicator (FIG. 15B). FIG. 15C shows a side view of the coating or covering strip positioned over the tampon device with a front view of the strip positioned over the tampon seen in FIG. 15D.

FIG. 16 shows a fiber coating or covering wherein in the fiber is either hanging, inserted through or attached to the tampon (FIG. 16A and FIG. 16B) or wound around the tampon (FIG. 16C and FIG. 16D).

FIG. 17A shows a strip coating or covering incorporated with a mucoadhesive composition comprising a therapeutic or health-promoting agent applied around the vaginal tampon, with an alternative arrangement where the strip is attached tot a string attached to the vaginal tampon (FIG. 17B). FIG. 17C shows a coating or covering cap placed around the bottom (distal end) of the vaginal tampon and the mesh placed as a cap over the top (proximal end) of the tampon seen in FIG. 17D.

FIG. 18 shows a vaginal device wherein the string attached to the vaginal tampon is impregnated with a coating comprising a mucoadhesive composition comprising a therapeutic or health-promoting agent wherein, seen in FIG. 18B, such coating is applied directly to the tampon. FIG. 18C shows alternative arrangement where the coating is attached to an applicator outer side.

FIG. 19A shows an arrangement where a mucoadhesive composition comprising a therapeutic or health-promoting agent is incorporated into the tablet or pellet sequestered within the tampon body and separated by a coating barrier so that the agent is released exclusively from the pellet or tablet (FIG. 19B). FIG. 19C shows a pellet attached to the tampon withing a coating cap. The pellet embedded in a swelling plug is seen in FIG. 19E and the pellet separated from the tampon with a swelling cap barrier is shown in FIG. 19F.

FIG. 20A shows a coating or covering of an applicator tube modified to be made of dissolvable material comprising a mucoadhesive composition comprising a therapeutic or health-promoting agent and its alternative wherein only a portion of the applicator is coated (FIG. 20B). FIG. 20C shows an arrangement wherein a mucoadhesive composition comprising a therapeutic or health-promoting agent is placed in a high density coating on the tip of the vaginal tampon and a variation thereof where there is a barrier coating placed in between the high density coating and between the tampon (FIG. 20D).

FIG. 21 shows exemplary arrangement of the coating or covering according to the invention. A homogeneous layer coating is seen in FIG. 21A, whereas FIG. 21B shows zoned deposits of a mucoadhesive composition comprising a therapeutic or health-promoting agent within an inactive substrate layer. FIG. 21C show a three-layer coating with the a mucoadhesive composition comprising a therapeutic or health-promoting agent incorporated into the middle layer or, as seen in FIG. 21D, active layers are positioned on the sides of the inactive layers. Another arrangement, shown in one layer coating is seen in FIG. 21E where the active portions of the coating layer are separated by the inactive layer or where the active layer is placed on top of the inactive layer as seen in FIG. 21F.

### DEFINITIONS

“Agent”, “active compound”, “chemical substance”, “active ingredient” or “drug” means a therapeutically effective compound suitable for treatment, management, or control of pathophysiological condition.

“Therapeutical agent” or “pharmacological agent” means a therapeutic agent, a health-enhancing agent, a mixture of both, or any other therapeutically effective pharmaceutically acceptable agent.

“Proximal end” means the end of the vaginal situated closest to the uterus and upper wall of the vagina when the tampon, tampon-like foam or another vaginal device is inserted therein.

“Distal end” means the end of the vaginal device situated away from the uterus and from the upper wall of the vagina when the tampon, tampon-like foam or another vaginal device is inserted into the vagina.

“Vaginal device” means a vaginal tampon, dissolving or non-dissolving, degradable or non-degradable vaginal foam, tampon-like foam or any other vaginal device, such as pessary, sponge, tablet, suppository, pellet, ring but also a vaginal applicator or any other structure generally insertable into vagina.

“Pharmaceutical ingredient” or “excipient” means a pharmacologically inactive pharmaceutically acceptable compound added to a mucoadhesive composition of the invention. The ingredient or excipient does not have any pharmacological properties.

“Rapid delivery” means initial immediate rapid release and delivery of the drug from the mucoadhesive composition. The rapid delivery is typically followed by a time-dependent reduction in release of the drug from the mucoadhesive composition or device and delivery of the drug to the plasma.

“Continuous delivery” means continuous and uninterrupted release of the drug from the formulation or device and delivering such drug in a continuous manner. Continuous delivery may be preceded by the rapid delivery.

“Pulsed delivery” means a release and delivery of the drug in intermittent intervals. Such pulsed delivery may be provided, for example, by formulating the drug in individual layers interspaced with inactive layers of dissolvable coatings or by using different pharmaceutical ingredients.

“Interesterified stone oil” means a vegetable oil ethoxylated by replacing part of glycerol of the glycerides contained in vegetable oil by polyoxyethylene glycols of different lengths. Such replacement results in hydrophilic properties. Example of the interesterified stone oil is LABRAFIL®, particularly LABRAFIL® M1944 CS, commercially available from Gattefossé.

“Mucosa” or “mucosal tissue” means surface epithelial tissue that is accessible from the outside of the body without surgical procedures.

“Mucosal composition” or “mucoadhesive composition” means a composition which is suitable for administration to the mucosal tissue and adhere to such mucosal tissue.

“Permeation enhancer” means a compound that promotes transfer of an agent across a mucosal barrier and is increasing the mass transfer of the agent to, into, as well as through the mucosal epithelium.

“Health-promoting agent”, “health-enhancing agent”, “health product” or “natural health product” means a substance or combinations of substances found in nature or energetically potentiated preparations that are used for the purpose of maintaining or improving health or treating or preventing disease conditions. These compounds include, but are not limited to, homeopathic preparations, vitamins, minerals, enzymes, co-enzymes, co-factors, herbs or botanicals, naturally occurring animals, plant and microorganism substances, and a variety of molecules extracted from natural sources such as amino acids, polysaccharides, peptides, naturally occurring hormones and biochemical intermediates, as well as naturally occurring molecules synthesized by chemical or biological means.

“Terpenoid” means natural secondary metabolite or its derivatives with a molecular structure containing a carbon backbone made up of 1-10 isoprene(2-methylbuta-1,3-diene) building units joined by head-to-tail and tail-to-tail condensation or a combination thereof. The basic C₅ₙ chain may be folded to produce rings or functionalized by introduction of heteroatoms, predominantly oxygen, which can be further modified by glycosylation.

“Alkaloid” means natural secondary metabolite or its derivatives with a molecular structure containing at least one nitrogen with the exception of amino acids, proteins, and nitrogen-containing polyketides. Most alkaloids are heterocyclic exhibiting weak basic properties.

“Shikimate” means natural secondary metabolite or its derivative that are synthesized via the shikimic acid pathway exhibiting a molecular structure with at least one phenolic hydroxyl group that can be glycosylated and/or esterified. Invididual shikimates can also condensate forming oligopolymers.

“Polyketide” means natural secondary metabolite or its derivative with a molecular structure containing a carbon backbone made by extension of a starter unit that can be as simple as acetyl-CoA or more complex such as the C₇-N units derived from aminoshikimate pathway, with a —CHR—CO— extender unit where R═H or alkyl, frequently —CH₃ or —C₂H₅.

### DETAILED DESCRIPTION OF THE INVENTION

The present invention is based on a discovery that a vaginal device, such as a tampon, tampon-like device, vaginal foam, vaginal pessary, vaginal ring, vaginal pellet, vaginal tablet, vaginal suppository or another type of vaginal device partly or completely coated, covered with a coating or combined with a covering comprising a film, foam, strip, foil, cap, cup or particle materials attached permanently or removably to the vaginal device as a layer, layers, cap, cup, suppository, tablet, pellet, soft gel capsule, strip or strips incorporated with a mucoadhesive composition comprising a therapeutic and/or palliative amount of an therapeutic and/or health-enhancing agent represents a suitable means for delivery of said agent to the uterus and/or to the general circulation for control and treatment of various pathophysiological conditions or for improvement of health.

The invention thus concerns a targeted delivery of therapeutical and/or health-enhancing agents to the uterus and/or to the general circulation using a vaginal device completely or at least partially coated or covered with a layer or layers of the coating material incorporated with or having attached to a mucoadhesive composition comprising an therapeutical and/or health-enhancing agent, or a combination thereof. The vaginal device allows delivery of the an therapeutical and/or health-enhancing agent directly to the uterus or to the general systemic circulation and permits a treatment of various pathophysiological conditions or enhances health in healthy individuals with lower concentrations of therapeutical agent or naturally occurring health product than those needed for oral administration and thus provides for lower systemic concentration and fewer side effects or, in alternative provides for a convenient delivery of the health-enhancing agent.

Additionally, the invention concerns the discovery that when the therapeutical or health-promoting agents are administered intra or transvaginally, preferably using an vaginal device of the invention for delivery of the therapeutical or health-enhancing agent, uterus allows for preferential uptake of the such agent into the uterus and into the general circulation thereby bypassing the first pass hepatic degradation and detoxification.

The current invention provides several previously unrecognized advantages. When a vaginal device coated according to the invention additionally contains an active layer containing the mucoadhesive composition comprising the therapeutical or health-enhancing agent, the released agent from the mucoadhesive composition is sequestered within the active layer of the coating or a portion thereof and prevented from being absorbed into the non-coated portion of the device thereby making the whole dose of the drug available for transmucosal absorption.

Additionally, when the mucoadhesive composition comprising the therapeutical or health-enhancing agent is incorporated into a layer of a temperature-sensitive coating, such as, for example, wax, the coating melts upon insertion into the vagina and releases the composition and the agent therefrom. Under these conditions, the mucoadhesive composition containing the entire dose of the agent is released and delivered to the upper vaginal wall closest to the uterus where, through its mucoadhesive properties, the composition adheres to said vaginal wall and the agent is transported through the vaginal wall to the uterus and/or to the general circulation.

The invention thus permits a quantitatively more efficacious delivery of the agent wherein the substantially whole dose of the agent formulated as a mucoadhesive composition and attached to or incorporated into the coating or a coating layer is released and delivered.

Transmucosal vaginal delivery systems according to the invention offers a viable alternative to deliver therapeutical and/or health-promoting agent to a female subject suffering from various pathophysiological condition or disease or desiring to improve and promote health. In addition, agents delivered transmucosally through vaginal mucosa enter the systemic circulations system bypassing the first pass liver detoxification and degradation. Consequently, this route of administration is particularly advantageous for agents, such as therapeutical and health-enhancing agents, that undergo substantial first-pass hepatic metabolism which deactivates a large portion of the agent. Furthermore, women are generally accustomed, on a routine basis, to the insertion of a vaginal device such as a tampon for menstrual control and are expected to embrace this alternative route of delivery for therapeutic uses or for health reasons without dramatic emotional distress.

The method of the invention provides a novel and more efficacious route of delivery of therapeutical and/or health-enhancing agent for treatment and control of the inflammation, pain, migraine, nausea, osteoporosis, vascular disorders, chemotherapy, radiotherapy, post-surgery pain or nausea, pre-operative medication, PMS, menstruation, pregnancy, breast feeding and menopause and other diseases and conditions as well as for delivery of the natural health promoting products including, for example, vitamins, herbs, minerals, enzymes, co-enzymes, factors, co-factors or immunoenhancers, where administration of these drugs provides relief of symptoms or enhances health in the female subjects. The method avoids drug administration to the gastrointestinal tract of patients, protects the therapeutic or health-promoting agent from extensive hepatic first-pass metabolism, permits rapid or slow, continuous or pulsed delivery of the therapeutical and/or health-enhancing agent, and achieves therapeutically effective concentrations of such agent in the blood circulation with much smaller amounts of the agent.

The method for drug delivery according to the invention comprises administration of a mucoadhesive composition containing a therapeutically effective amount of the appropriate therapeutical agent or desired health-enhancing agent incorporated into a vaginal device of the invention. The mucoadhesive composition attached to or incorporated into a coating or covering at least a portion of the vaginal device is introduced into vagina attached to the vaginal device and brought into a close contact with a vaginal mucosa for direct absorption and transport through the mucosa into uterus and/or to the systemic circulation.

Administration of therapeutical and/or health-enhancing agent via the vaginal route as described herein reduces the portion of the drug dose which would be eliminated by the liver during its first pass circulation in the blood system, which further enhances the drug's therapeutic effect.

I. Methods, Device and Compositions for Vaginal Delivery of Therapeutical or Health-Promoting Agents

A method for vaginal delivery of therapeutical health-enhancing agent comprises preparation of a vaginal device completely or at least partially coated with a layer or layers of coating or covering in the form of a film, foam, strip, cap, cup or particles or is in combination with a covering in the form of film, foam, strip, cap, cup, tablet, soft gel capsule, pellet or suppository that is incorporated with or having attached thereto a mucoadhesive composition comprising at least one therapeutical or one health-enhancing agent, alone or in combination with other therapeutical agents and health-promoting agents, or with each other. The mucoadhesive composition may contain a mixture of therapeutical and health-enhancing agents and may additionally and optionally also contain other therapeutical or health-promoting agents and/or other pharmaceutically acceptable excipients.

The mucoadhesive composition typically contains at least one mucoadhesive agent permitting the adhesion of the composition to the vaginal wall for a time needed for the active therapeutical and/or health-enhancing agent to be absorbed through the vaginal mucosa into the uterus and/or general systemic circulation. Such delivery of the therapeutical and/or health-enhancing agent occurs without oral administration and thus eliminates secondary symptoms and undesirable reaction typically occurring with oral administration of some therapeutical agent and eliminates oral delivery of health-enhancing agent that are often formulated as large capsules or tablets.

A. Advantages of Vaginal Delivery

Existing therapeutic approaches used to deliver therapeutic agents mostly depend on oral, intravenous, nasal or rectal drug delivery systems. Unfortunately, drug administration via the gastrointestinal tract is often very inefficacious. Alternatively, parenteral intramuscular or subcutaneous injections, nasal sprays, or insertion of rectal suppositories are employed to bypass efficacy problems and difficulties encountered with oral administration of these agents to patients. In this regard, injection methods usually require visit to a medical facility and assistance of a trained health care professional, whereas many patients find insertion of a rectal dosage form uncomfortable and/or emotionally unpleasant. Nasal delivery systems have been only partially successful as the drug dose needed to achieve a relief from, for example, pain or nausea, needs to be adjusted to consider first pass liver deactivation of the substantial amount of the agent and thus is efficacious only for drugs that are highly resistant to hepatic metabolism.

The vaginal route of delivery allows rapid or slow, continuous or pulsed delivery of the therapeutical and/or health-promoting agents in a patient-controlled environment without the need to have access to a skilled health care professional in a doctor's office or hospital. Using the mucoadhesive composition and a vaginal device of the invention, an effective dose of a desired therapeutical agent or health product can be delivered reproducibly to the systemic circulation and eliminates parenteral injection with all its adverse effects and requirements. Furthermore, since the blood circulation into which the agents are delivered through vaginal mucosa circumvents the liver first-pass circulation, the dose of the vaginally delivered agent is substantially smaller compared to the portion of the agent administered orally. In this regard, the vaginal delivery is many times more efficacious.

The invention, thus, concerns discovery of an improved delivery route of therapeutical and health-enhancing agent that overcomes the side effects and limitations observed during oral, parenteral, and nasal administration of these agents in subjects suffering from various conditions. The invention utilized anatomic advantage of female subjects by focusing the delivery of therapy directly to the vaginal mucosa using a specifically formulated mucoadhesive composition attached to or incorporated into a coating of a vaginal device.

The newly developed vaginal delivery strategy of therapeutical and/or health-enhancing agent according to the invention, therefore, represents an important improvement in the systemic delivery of these agents and an important advancement of therapy generally as well as the delivery of the health-promoting natural products.

B. Therapeutical and Health-Enhancing Agent

Vaginal delivery of therapeutical and/or health-enhancing agent comprises formulating said therapeutical or health-enhancing agent into a mucoadhesive composition, incorporating said composition or attaching it to a layer, layers, strip, cup, cap, particles forming a coating of at least a portion of a vaginal device or incorporating said composition into a covering as a layer, layers, strip, cup, cap, pellet, suppository or soft gel capsule and introducing said vaginal device into the vagina.

Examples of therapeutical agents suitable to be used in the current invention are representative therapeutical agents selected from the group consisting of an antimicrobial, vasodilator, nonsteroidal anti-inflammatory (NSAI), prostaglandin inhibitor, COX-1 inhibitor, COX-2 inhibitor, local anesthetic, calcium channel antagonist, potassium channel blocker, β-adrenergic agonist, bisphosphonate, leukotriene blocker, smooth muscle inhibitor, peptide, protein, dyskinetic muscle contraction inhibitor and anti-HIV agent and a combination thereof.

The representative antimicrobial agent is selected from the group consisting of acyclovir, afloxam, amantadine, amphothericin B, azitormycin, bacampicillin, butoconazole, carbenicillin, cefadroxil, cefixime, ceflotoxime, cefpodoxime, cefprozil, cephalexin, cephradine, ciclopirox, ciprofloxacin, clidamycin, clotrimazole, dirithromycin, dosicycline, doxycycline, econazole, erythromycin, famciclovir, fenticonazole, fluconazole, flucytosine, ganciclovir, isoconazole, itraconazole, ketoconazole, lumefloxacin, metronidazole, miconazole, mupirocin, naftifine, norfloxacin, nystatin, oseltamivir, oxiconazole, penciclovir, phosphomycin, ribavirin, rimantidine, sulconazole, terconazole, tetramycin, tioconazole, troleandomycin, voriconazole, and zanamivir.

The representative non-steroidal anti-inflammatory agent including COX-1 or COX-2 inhibitors is selected from the group consisting of acetaminophen, acetylsalicylic acid, bromfenac, celecoxib, darbufelone, diclofenac, diflunisal, etodolac, etoricoxib, fenamate, fenoprofen, flosulide, flurbiprofen, ibuprofen, indomethacin, ketoprofen, ketorolac, lumiracoxib, meclofenamate, meloxicam, nabumetone, naproxen, nimesulide, oxaprozin, parecoxib, phenylbutozone, piroxicam, rofecoxib, salsalate, sulindac, teroxicam, tolmetin, and valdecoxib.

The representative calcium channel antagonist is selected from the group consisting of amlodipine, bepridil, diltiazem, felodipine, israpidine, nicardipine, nifedipine, nimodipine, and verapamil.

The representative potassium channel blocker is selected from the group consisting of 4-aminopyridine, almikalant, ambasilide, amiodarone, apamin, azimilide, charybdotoxin, clofilium, clotrimazole, correolide, dequalinium chloride, dofetilide, glibenclamide, glyburide, ibutilide, paxilline, procain, sematilide, sotalol, tedisamil, tetramethylammonium, and tolazamide.

The representative beta-adrenergic agonist is selected from the group consisting of formoterol, levalbuterol, metaproterenol, pirbuterol, ritodrine, salbutamaol, salmeterol, and terbutaline.

The representative vasodilator is selected from the group consisting of clonidine, dinitrate, doxazosin, guanabenz, guanfacine, hydralazine, isosorbide isosorbide mononitrate, isosorbide dinitrate, methyldopa, minoxidil, nitroglycerin, prazosin, rilmenidine and terazosin.

The representative bisphosphonate is selected from the group consisting of alendronate, alpadronate, clodronate, etidronate, ibandronate, neridronate, olpadronate, pamidronate, residronate, tiludronate, and zoledronate.

The representative anti-migraine agent is selected from the group consisting of ergotamine, dihydroergotamine, ergostine, butalbital, phenobarbital, acetaminophen, diclofenac sodium, ketoprofen, ketorolac, ibuprofen, piroxicam, naproxen, acetylsalicylic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate and divalproex sodium.

The represenative anti-nausea agent is selected from the group consisting of metoclopramide, palonoestrol, gabapentin, olanzapine, doxylamine, prochlorperazine, domperidone, ondansetron, tropisetron, dolasetron, nabilone, dronabinol, levonantradol, aprepitant, cyclizine and promethazine.

The representative anti-cancer agent is selected from the group consisting of alitretinoid, altretamine, anastrozole, bexarotene, bicalutamide, bisulfan, capecitabine, chlorambucil, cisplatin, docitaxel, doxorubicin, estramustine, etoposide, exemestane, gefitinib, gemcitabine, imatinib, irinotecan, letrozole, lomustine, melphalan, methotrexate, nilutamide, paclitaxel, procarbazine, tamoxifen, temozolomide, thioguanine, topotecan, toremifene, tretinoid and vincristine.

The representative anti-HIV agent is selected from the group consisting of abacavir, amprenavir, atazanavir, delavirdine, didanosine, efavirenz, emtricitabine, enfuvirtide, fosamprenavir, indinavir, lamivudine, lopinavir, nelfinavir, nevirapine, ritonavir, saquinavir, stavudine, tenofovir, zalcitabine, and zidovudine.

The representative protein or peptide is selected from the group consisting of insulin, parathyroid hormone, calcitonin, vasopressin, oxytocin, interleukin, immunoglobulin A, immunoglobulin G, monoclonal antibodies, oxytocin, humanized antibodies, human growth hormone and a fragment thereof.

It is to be understood that all therapeutical agents of the same or similar type are intended to be covered by this invention.

C. Natural Health-Promoting Agents and Products

Natural health products are substances or combination of substances produced by plants, animals, and microorganisms that exhibit beneficial medical or cosmetic effects in humans when used appropriately.

In the context of female reproductive health, this includes maintaining or improving physiological health status as well as treating or preventing specific disease conditions associated with female reproductive organs.

Plants, animals, and microorganisms produce a diverse array of chemical compounds that are chemically based on carbon. These chemical products can be classified into primary and secondary metabolites.

Primary metabolites are those common to a large variety of species and include the groups of proteins, carbohydrates, lipids, and nucleic acids. In contrast, secondary metabolites are not ubiquitously present in most organisms but rather specifically produced in a few or even only in one species. Often, they are only generated during a limited developmental period of the organism.

The chemical classification of secondary metabolites can be based on the synthetic pathways by which they are produced in the plant, animal, or microorganism. For the purpose of this invention, the four major categories that include important examples of natural health products with relevant applications in female reproductive health are described below.

Terpenoids are formed by a wide variety of plants, animals and microorganisms. The role of these metabolites for the living organism can be functional, such as d-α-tocopherol that prevents oxidative damage to the cells, can be part of the defense against predators, such as resins, or used as chemical messengers, such as for example, pheromones. Medical applications of terpenoids span a wide variety of pharmacological targets. Particularly relevant for maintaining or improving vaginal health are terpenoid-associated actions on blood flow, inflammation, oxidation, growth of microorganism, and epithelial function. In addition, volatile terpenoids find cosmetic applications as fragrances while resins can be used as emollients and astringents, respectively.

Alkaloids are a large group of nitrogen-containing secondary metabolites of plant, animal, or microbial origin. Biogenetically and structurally the alkaloids are diverse but can be organized according to the nitrogen source included in those compounds. Consequently, groups of alkaloids are derived from ornithine, lysine, nicotinic acid, polyketides, anthranilic acid, phenylalanine, tyrosine, lysine, and tryptophane. Many constituents of plants and other natural sources that belong chemically to the class of alkaloids have demonstrated pharmacological and, in some cases, also toxic effects. Alkaloids such as 6,8-didec-(1Z)-enyl-5,7-dimethyl-2,3-dihydro-1H-indolizinium, which is isolated from Aniba panurensis (Meissn.), or berberine found in the stem bark of Mahonia aquifolium (Pursh.) are examples of natural health products that have potential applications in vaginal health due to their antimicrobial activity.

Shikimates are phenolic compounds that directly or indirectly result from degradation of shikimic acid, which is a key intermediate in plants and microorganisms. Particularly relevant to medical applications of shikimates are phenolic compounds summarized as isoflavones, lignins, coumarins, and tannins, which can be glycosylated to various degrees. Members of those important subgroups exhibit numerous beneficial pharmacological activities, including cardiovascular, antimicrobial, antioxidant, antiallergic, immunostimulating, antineoplastic, and astringent. Therapeutically used examples include (+) epigallocatechin, quercetin, and resveratrol all present in tea and red wines as well as apigenin and cyanidin-3-glucoside isolated from Medicago sativa (L.) and Ribes grossularia (L.), respectively.

Polyketides represent a class of diverse natural health products that originate from a single starter unit but vary in chemical complexity, through enzymatic elongation mediated by polyketide synthases usually with malonic acid or a substituted malonic acid thioester under loss of CO₂. Small units can further polymerize in more complex structures into macromolecules, including fatty acids nucleic acids, polysaccarides, and proteins. Therapeutically, polyketides are valuable in cardiovascular diseases, infections, and spasms. Halogenated polyketide particularly from Laurencia ssp. have demonstrated potent antimicrobial and cytotoxic properties.

Medical applications of natural health products described above includes delivering or administering whole organisms, parts of the organism, molecules extracted from the organism, and/or purified, chemically identified constituents of plants, animals, or microorganisms. The vaginal cavity of healthy women is normally colonized with resident Lactobacillus microorganisms that prevent adhesion and growth of pathogenic microorganisms through mechanisms that appear to involve secretion of lactic acid, anti-adhesion factors, hydrogen peroxide, and bacteriocins lethal to pathogens (Microb. Infect., 4, 319-324(2002)). Following the success of pre- and probiotic strategies in various gastrointestinal disease states (Curr. Opin. Gastroenterol., 21:44-50 (2005)), vaginal administration of suitable Lactobacillus strains will improve health conditions that result from imbalanced vaginal microflora. To favor the growth of beneficial Lactobacillus strains over pathogenic microorganisms, including Escherichia coli, Enterococcus faecalis, Proteus mirabilis, Staphylococcus aureus, and Candida albicans, probiotic agents such as natural amino acids, ascorbic acid, D-pantothenic acid, folic acid, niacinamide, p-amionbenzoic acid, and ethylenediamine tetraacetate are incorporated into a suitable vaginal device.

For the purpose of maintaining or improving physiological health or treating or preventing disease conditions in the female, a diverse array of plant constituents can be used, which are chemically defined by the four classes of terpenoids, alkaloids, shikimates, and polyketides. Since secondary metabolites are generally sequestered in particular areas of the organism, extracts of only parts of the plant are administered to achieve the expected health benefit. This can include fresh or dried organs of the plant such as the berries of Vitex agnus-castus (L.), the leaf latex from Aloe vera (L.), the nut of Areca catechu (L.), the rhizome of Cimicifuga racemosa (L.), the herb of Equisetum arvensis (L.), or the bark of Magnolia officinalis (L.), respectively.

The constituents of interest are extracted using appropriate technologies known to the skilled in the art starting from simple infusion with water to more complex supercritical extraction methods. The extracted constituents can be further purified and processed to improve the desired health benefit. This includes enhancement of potency applying the classical principles of homeopathy (J. Obstet. Gynecol. Neonatal Nurs., 32: 207-214 (2003). Where appropriate, pure plant constituent alone or in a mixture can be incorporated into the device of this invention with the objective to achieve the desired health benefit. One such example is menthol(2-isopropyl-5-methyl-cyclohexan-1-ol), which is a major component in various Mentha species, that is therapeutically used as an anti-itching agent with cooling and mild anti-inflammatory activities. Although originally isolated from natural sources, it is now chemically synthesized to meet the world demand.

Thus the vaginal device of the invention is coated with or covered or combined with a covering comprising a mucoadhesive composition comprising at least one health-promoting agent selected from the group consisting of a botanical, probiotic microorganism, vitamin, herb, enzyme, co-enzyme, factor, co-factor, antioxidant, anti-pruritic additive and synergistic additive agent and a combination thereof.

The representative health-promoting agent is a botanical selected from the group consisting of Agnus castus, aloe vera, comfrey, calendula, dong quai, black cohosh, chamomile, evening primrose, Hypericum perforatum, black currant seed oil, St. John's wort, tea extracts, lemon balm, capsicum, rosemary, Areca catechu, mung bean, borage seed oil, witch hazel, fenugreek, lavender, soy, Vaccinium extract, heath, azaleas, red onion skin, beat root extract, capsanthin and capsaicin.

The representative terpenoid is selected from the group consisting of 1,8-cineole, agnostinde, aucubin, harpagide, α- and β-pinene, manoalide, oleuropein, vitexin, luteolin 7-O-glucoside, rotundifuran, vitexilactone, casticin, isovitexin, orientin, 6β,7β-diacetoxy-13-hydroxy-λ-8-14-diene, vitexilactone, altissinone, 2″-O-p-hydroxybenzoylorientin, euscaphic acid glucoside ester, γ-linolenic acid, actein, 23-epi-26-deoxyactein, cimiracemoside A.

The representative alkaloid is selected from the group consisting of arecoline, arecain, guracine, lobeline, papuamine, bastidin, morphine, atropine and vincristine.

The representative aliphatic, aromatic, or heteroaromatic organic acid is selected from the group consisting of ursolic acid, corosolic acid, epicorosolic acid, maslinic acid, epimaslinic acid, euscaphic acid, gallic acid and caffeic acid.

The representative phenol is selected from the group consisting of aloin A, aloin B, 7-hydroxyaloin, tannin, gallotannin and menthol.

The representative polyketide is selected from the group consisting of acemannan, spiramicyn, nystatin, erythromycin, lovastatin, doxorubicin, maytansine and brevetoxin.

The representative iridoid is selected from the group consisting of agnoside and aucubin.

The representative volatile oil, resin or balm is selected from the group consisting of aloeresin A and aloeresin B.

The representative amino acid is a natural amino acid.

The representative mineral is selected from the group consisting of calcium, chromium, iron, magnesium, manganese, potassium, selenium and zinc.

The representative vitamin is selected from the group consisting of riboflavin, thiamine, β-carotene, cyanocobalamine, pyridoxine, ascorbic acid, cholecalciferol and d-α-tocopherole.

The representative co-enzyme/factor is selected from the group consisting of biotin, choline, folic acid, D-pantothenic acid, lecithin and niacin.

The representative probiotic microorganism is selected from the group consisting of Lactobacillus acidophilus CRL1259, Lactobacillus brevis CRL1335, Lactobacillus crispatus CTV05, Lactobacillus fermentum RC-14, Lactobacillus rhamnosus GR-1 and Lactobacillus salivarius CRL1328.

The representative synergistic additive is selected from the group consisting of caffeine and ethyoxydiglycol.

D. Formulating Agents and Dose Amounts

Each therapeutical or health-enhancing agent may be formulated and delivered alone or in combination with each other or with another therapeutically effective agent or with a pharmaceutically acceptable excipient. Typically, the agent will be formulated in combination with at least one mucoadhesive agent, one carrier and one penetration agent.

The therapeutical agent is present in a dose sufficient to assert its therapeutic effect, typically from about 0.00001 to about 45 mg/kg body weight, preferably from 0.001 to 15 mg/kg body weight, most preferably from 0.1 to 8 mg/kg body weight. The health-enhancing agent may be present in a maximum formulating capacity, typically will be present in from about 0.00001 to about 100 mg/kg body weight, depending on the agent.

E. Confirmation of Vaginal Delivery of Therapeutical and Health-Enhancing Agent

Transmucosal delivery of therapeutical agents across the vaginal mucosa results in therapeutically useful systemic plasma concentrations. Consequently, vaginal administration represents a viable alternative to oral dosing for pharmacological agents with beneficial effects in the treatment of various disease and conditions.

To confirm feasibility of the method according to the invention, a series of in vivo pharmacokinetic studies was performed using female white New Zealand rabbits. Following administration of the therapeutical drugs sumatriptan and the metoclopramide as a solution intravenously by injection into the marginal ear vein or via the oral route using a rubber tubing inserted into the stomach as well as vaginally whereby the drug was incorporated into a suppository, plasma samples were withdrawn from the animal at predetermined time points and the drug concentration was quantitatively analyzed applying sensitive analytical methodologies.

Model-dependent pharmacokinetic analysis was further utilized to calculate relevant pharmacokinetic parameters such as maximum plasma concentration (cₘₐₓ), time required to reach maximum plasma concentration (tₘₐₓ), total exposure of the body to the drug extrapolated to infinity (AUC_{∞}), and elimination half-life (t_{1/2}). All studies were repeated at least three times in different animals, and pharmacokinetic calculations were performed using WinNonlin 4.1.

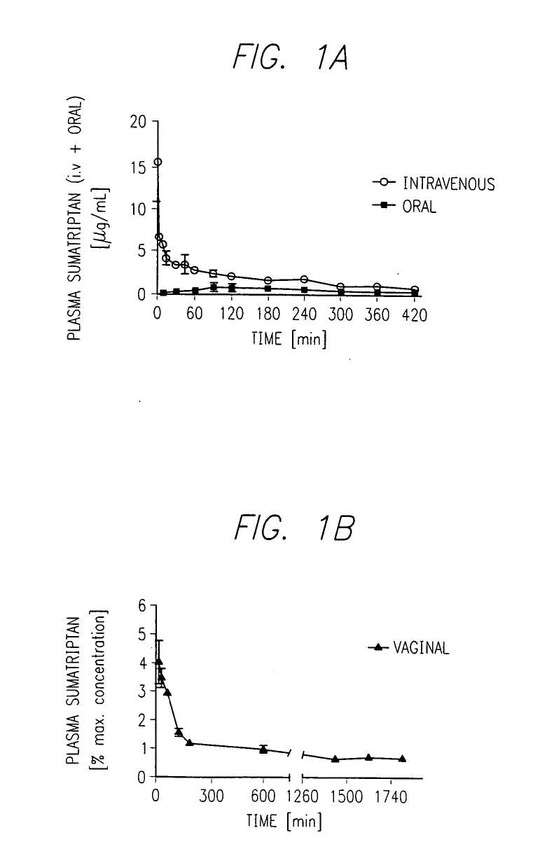

The results for the sumatriptan are shown in FIGS. 1A and 1B.

FIG. 1A is a graph illustrating pharmacokinetic of an sumatriptan in female white New Zealand rabbits following vaginal administration. Each animal received 0.7 mg of sumatriptan per kg body weight administered as a solution. Open circles (◯) denote plasma concentration following intravenous injection, whereas closed squares (▪) represent corresponding drug concentration measured in the plasma after oral dosing of the drug solution. Experiments were performed in three different animals and results are presented as means±S.D. FIG. 1B shows systemic plasma concentrations of sumatriptan in female white New Zealand rabbits after vaginal insertion of a lipophilic delivery device. Each animal received 0.7 mg of sumatriptan per kg body weight and plasma concentrations were normalized to cₘₐₓ measured in similar experiments after oral administration of a dose-equivalent sumatriptan solution. Experiments were performed in three different animals and results were presented as means±S.D. In these studies, a dose of 0.7 mg sumatriptan per kg body weight of the animal was used. For analytical purpose, the dose was supplemented with a trace amount of [³H]sumatriptan, which was used to quantify plasma drug concentrations by liquid scintillation counting. Results are presented as means±SD.

FIG. 1A demonstrates that parenteral injection of the drug solution results almost immediately in high plasma concentrations that rapidly decrease with an apparent t_{1/2} of 191±6 min. Therapeutically, the initial high concentrations with a cₘₐₓ of 15.4±4.5 μg/mL suggest fast onset of an effective relief of migraine headache and associated symptoms. However, since the drug is administered as a bolus into the blood stream, there is no constant supply that would support a longer duration of the action. Elimination of sumatriptan from the systemic circulation through metabolism and renal excretion are the predominant mechanisms by which the time of the therapeutical effect is determined after injection.

In contrast, oral administration results in a very slow increase of systemic plasma concentrations of sumatriptan in the rabbit. This underlines kinetically the absorption phase required for the drug to physically move from the gastrointestinal tract across the epithelial barrier into the blood vessels of the submucosa. Physicochemical properties of the drug molecule, including ionization, lipophilicity, and hydrodynamic radius determine the rate of absorption.

Comparison of the two systemic profiles clearly suggests that oral administration of sumatriptan will result in a delayed therapeutical effect as compared to parenteral injection. The tₘₐₓ values estimated for both i.v. and oral routes of administered drug indicate that the maximum effect of sumatriptan after oral administration is delayed by at least a factor of 100, which is therapeutically a dramatic disadvantage.

The overall exposure of the body to the same dose administered via the intravenous and the oral routes are significantly different as shown by the respective total area under the time/plasma concentration curve. The AUC_{∞} calculated for oral administration is 958±163 μg×min/mL and 10868±90 μg×min/mL (approximately 11 times higher) for parenteral administration, respectively. The apparent t_{1/2} of 1210±163 minutes for oral administration is significantly longer than calculated from the intravenous data, which suggests that absorption rather than elimination becomes pharmacokinetically the rate-limiting process for this drug after oral administration.

To contrast the therapeutic potential of vaginal administration of therapeutical agents to the more frequently used oral route of administration, systemic plasma levels of sumatriptan measured after insertion of lipophilic vaginal device comprising sumatriptan composition were normalized to the maximum concentration attained after oral administration. Results are seen in FIG. 1B. In this representation, a straight line indicates similar kinetic processes involved in the absorption of this drug across the vaginal and oral mucosa, respectively. However, the early peak in this profile strongly suggests that vaginal administration of sumatriptan using said vaginal device as described in this application provides more rapid delivery of the drug into the systemic circulation than traditional oral administration. The calculated tₘₐₓ for vaginal administration is ˜15 min, which implies that this delivery method may provide almost 6 times faster onset of therapeutical efficacy when compared to the oral route. This therapeutic benefit could be compared with the previously described bolus effect after intravenous injection. The total body exposure to sumatriptan after an equivalent dose is <5% of that measured after oral administration. This requires incorporation of greater amounts of therapeutical agent into vaginal delivery device than for oral delivery methods due to incomplete absorption. The AUC_{∞} calculated for 0.7 mg/kg after vaginal delivery was 23.0±0.1 μg×min/mL.

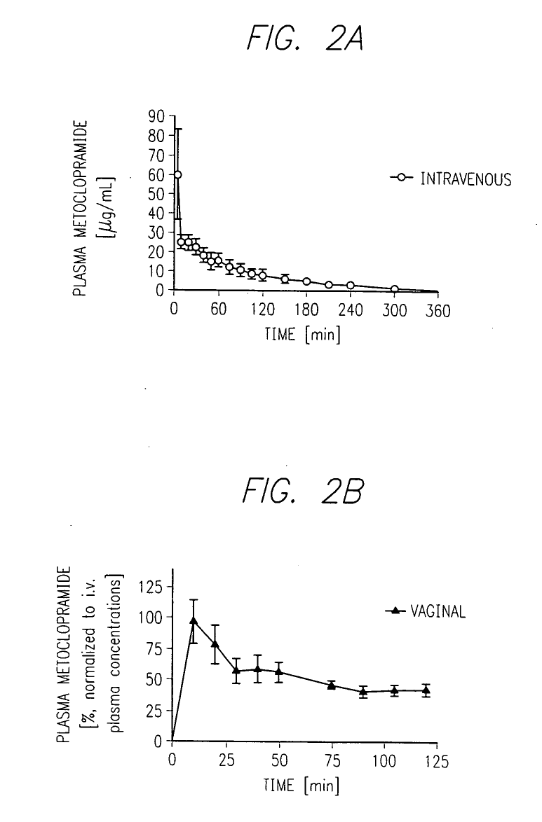

Similar studies as performed with the sumatriptan were performed to demonstrate the value of vaginal delivery of anti-nausea agent. Metoclopramide was selected as a model drug for this therapeutic class. Results are seen in FIGS. 2A and 2B.

FIG. 2A shows metoclopramide plasma concentrations in female white New Zealand rabbits after intravenous administration. Drug solution of 0.5 mg was prepared in sterile saline (0.9%; w/w) and injected into the systemic circulation through the marginal ear vein. Plasma samples were collected for six hours and analyzed for the drug using HPLC. Results are presented as means plasma±SEM (n=4). FIG. 2B shows normalized metoclopramide plasma concentrations in white female New Zealand rabbits after vaginal administration. Plasma samples were analyzed for the drug using a selective HPLC method and normalized to metoclopramide concentrations measured at the same time points after intravenous injection. Experiments were performed in three individual animals and results are presented as mean±SEM.

Three female white New Zealand rabbits were dosed at 0.05-0.1 mg/kg body weight intravenously, orally, and vaginally. Blood samples were removed at various time points, and plasma concentrations were quantified using a selective HPLC method described in Int. J. Clin. Pharmacol. Ther., 40: 169-174 (2002). Model-dependent pharmacokinetic parameters were calculated from time/plasma concentration data using WinNonlin 4.1.

Intravenous injection of a metoclopramide solution prepared in 0.9% saline immediately reaches average plasma concentrations around 60 ng/mL (FIG. 2A). First-order distribution and elimination phases result in a monophasic decline of plasma concentrations with an apparent t_{1/2} of 84±38 min. Total body exposure as measured by the AUC_{∞} was 3845±1415 ng×min/mL. Oral administration of the same dose resulted in peak plasma concentration that were on average 30-fold lower than measured after parenteral administration (data not shown). This implied that the transfer of this drug from the gastrointestinal tract into the systemic circulation is inefficient. Considering the additional complication that patients who are vomiting experience great difficulties, in general, to swallow oral dosage forms, it becomes apparent that oral delivery of anti-nausea drugs such as metoclopramide appears therapeutically undesirable. However, inclusion of this drug into a vaginal delivery device consisting of a lipophilic base such as SUPPOCIRE AS2, the mucoadhesive hydroxypropyl methylcellulose, and the permeation enhancer ethoxydiglycol provides a unique opportunity to bypass the irritated gastrointestinal tract in female patents and deliver therapeutically sufficient amounts into the systemic circulation.

Results of the vaginally administered metoclopramide depicted in FIG. 2B demonstrate that plasma levels of metoclopramide delivered vaginally using a device such as described above are close to the respective drug levels measured after intravenous injection. The profile shown in FIG. 2B was generated by normalizing the actual plasma levels measured for metoclopramide after vaginal administration to the time-equivalent levels after parenteral administration. Consequently, the initial concentrations appearing after 10 minutes following vaginal dosing are equivalent within subject variability to plasma levels after parenteral administration. Therapeutically, this may offer great benefit to the patient suffering from vomiting without the requirement to inject the drug.

The above described studies clearly demonstrate feasibility and benefits of vaginally delivered therapeutical agents. Clearly, such vaginal delivery provides much larger dosage of the agent delivered more rapidly and for longer time than that achieved by oral administration. Advantages of the vaginal delivery of therapeutical or health-enhancing agent compared to requirements, inconveniences and invasiveness of the intravenous administration are obvious.

II. Devices for Vaginal Delivery of Therapeutical and Health-Enhancing Agents

The vaginal device of the invention, such as a vaginal tampon, vaginal tampon-like device, vaginal foam, vaginal sponge, vaginal pessary, vaginal suppository, vaginal tablet, vaginal pellet or vaginal ring, provides an improvement against previously described devices. In particular, the device of the invention, which is preferably a degradable or non-degradable vaginal tampon or tampon-like device is coated completely or, preferably, only partly at its proximal or distal end or in the middle with a layer or layers of a coating, covering or is combined with such covering. The coating may be in the form of a film, foam, strip, cup, cap or particle or it may be a covering in the form of a foam, film, strip, cap, cup or pellet, tablet or suppository attached, as described or illustrated in the figures.

The material may be applied to the device as one layer or several layers interspaced with a layer or layers of different material, it may form a cap or cup covering a proximal or distal portion of the tampon or a strip, string or rim of the coating encircling the tampon. Since the vaginal tampon or vaginal foam is made of porous material, usually a cotton or polymer, the coating material covering at least a proximal portion, typically the proximal end of the tampon, separates the porous material from the material coated with the coating layer and sequesters the portion of such porous material from the portion comprising the therapeutical or health-promoting agent within the coating. The coating, whether the layer, layers, strip, strips, cap or cup, foam or film is incorporated with a mucoadhesive composition comprising a therapeutical or health-promoting agent or such composition is attached to such coating by various means.

The coating of the entire device prevents the absorption of the mucoadhesive composition into the porous portion of the device. The partial coating of the device permits sequestration of the drug to a smaller area and prevents the absorption of the mucoadhesive composition into the porous portion of the device. Thus, the loss of the drug due to reabsorption into the porous portion of the device is either eliminated or substantially decreased. Additionally, since the mucoadhesive composition comprising the therapeutical or health-enhancing agent is sequestered within the coating applied to the proximal end of the device, it is preferentially released from the device into the vicinity of uterus where the mucosal epithelia is more apt to absorb the agent.

The drug is therefore delivered more quantitatively to the vaginal mucosa to which it adheres due to the presence of the mucosal agent and is transported through the mucosa to the uterus and/or to the general systemic circulation due to the presence of the sorption promoter and/or penetration enhancer. The lipophilic or hydrophilic carrier additionally modifies the drug affinity to the mucosal surface and enhances the drug surface exposure.

A. Coated Vaginal Devices

The vaginal device of the invention is a vaginal tampon, dissolving or non-dissolving, degradable or non-degradable vaginal tampon or tampon-like shaped device, such as a foam, vaginal foam, vaginal sponge, vaginal ring, vaginal suppository, vaginal tablet, vaginal pellet or vaginal pessary, all coated or at least partially coated with a layer of coating separating the body of the device from the mucosal composition incorporated into or attached to said coating. The most preferred embodiment is a vaginal tampon or the tampon-like shaped device or foam.

1. Vaginal Tampon

One preferred embodiment for vaginal drug delivery is the vaginal tampon. The vaginal tampon is typically a commercially available vaginal tampon that is coated, according to the invention, either completely or partially, typically to about one third or one half, that is a portion coming in contact with the vaginal wall. The proximal or distal end, or a middle portion of the tampon is coated with a coating forming a layer, layers, cap, cup, film, foam, particles or strip around the upper proximal top portion of the tampon or attached to the tampon as a covering in the form of a cap, cup, strip, foam, film, tablet, suppository, soft gel capsule or pellet prepared separately. However, the whole tampon may also be coated with the coating, if desirable and the composition is then attached to the whole, to the proximate or distal part, or to the tip of the tampon.

2. Vaginal Foam

Another preferred embodiment is a tampon-like shaped vaginal foam that may be fully or partially dissolving or non-dissolving or degradable in the vagina or it may be non-degradable. However, the foam may also be shaped differently than a tampon-like structure.

The foam used as a vaginal device is preformed into a specific shape of a solid structure or a semi-solid or liquid preparation. The latter two may be used as a receptacle for the mucoadhesive composition which is applied in a form of a foam, film or particle layer, strip, cup or cap coating into which the composition may be conveniently incorporated.

The vaginal foams, as well as films, whether degradable or non-degradable and whether used as a vaginal device or a coating therefore, are prepared by processes known in the art that introduce porosity in a polymer matrix, namely by lyophilization, aeration, freeze drying, hydrocarbon templating, salt or particulate leaching, gel or solvent casting, gas expansion, sintering, polymerization of high internal phase emulsions, and free form fabrication techniques such as three-dimensional polymer printing.

The most preferred process to fabricate foams is lyophilization, which is described in detail in the copending application Ser. No. 10/600,849 filed Jun. 30, 2003. Lyophilized foams are open cell, high-surface-area, biodegradable or non-degradable constructs that can be manufactured from a variety of polymers, preferably from hydrophilic polymers. The foam materials are characterized by controlled chemical and physical properties that can be tailored according to their intended application. Tuneable properties include hydrophilicity, rate of absorption, degradation profile and dissolution rate, a measure of which is the time needed to complete dissolution of the foam.

Typically, the lyophilized foam is prepared by dissolving an appropriate polymer, preferably a hydrophilic polymer, or a mixture thereof, serving as a substrate material, as listed below, in an amount needed to prepare solution from 1 to 10% (w/w) in an aqueous or non-aqueous solvent, such as methanol, ethanol, glycerine, methylene, chloride, propylene glycol, propylene carbonate, glycofurol, cetyl alcohol, difluroethane and isopropylalcohol, preferably a purified water.

Alternatively, polymeric solutions with the drug and additives may be prepared in acetic acid, cyclohexane, acetonitrile, tert-butanol, ethanol, and isopropanol or in mixtures of aqueous and non-aqueous solvents.

Substrate materials for preparation of foam compositions of the invention are hydrophobic or, preferably, hydrophilic polymers. These polymers may be used singly or in combination with each other. They may be used in variable concentrations and ratios to each other when in admixture of two or several polymers.

Non-exclusive list of substrate polymers comprises cellulose and cellulose derivatives, microcrystalline cellulose, polyacrylic acid, polyethylene glycol, polypropylene glycol, divinyl glycol, polyethylene oxide, polypropylene oxide. Other possible polymers include the cellulose derivatives such as carboxymethyl cellulose, hydroxyethyl cellulose, polylactide, polyglycolide, polymethacrylic acid, poly-γ-benzyl-L-glutamate, polypropylene fumarate, poly-e-caprolactone, poly-butylene terephthalate, polyvinyl alcohol, polyvinyl ether, poly-1-vinyl-2-pyrrolidinone, 2,5-dimethyl-1,5-hexadiene, divinyl benzene, polystyrene-divinyl benzene, polyanhydrides such as poly-bis-p-carboxy-phenoxypropane-co-sebacic acid, polyhydroxyalkanoates such as poly-β-hydroxybutyrate or poly-β-butyrolactone, and alkyl-substituted silica gel such as tetraethylorthosilicate and dimethyldiethoxysilane.

Examples of hydrophilic polymers suitable for a foam manufacture include hydroxypropyl methylcellulose (HPMC), sodium carboxymethylcellulose, polyethylene glycol (PEG), alginic acid, alginic acid sodium salt, pectin, gelatin, collagen, polyvinyl pyrrolidone, poloxamer, acrylic-acid based polymers, such as carbopol, noveon, polyurethanes, polyvinyl alcohol, chitosan, hydroxypropyl cellulose, polyethylene oxide, fibronectin, hyaluronic acid, polysaccharide gums such as karaya gum, polyacrylamide, polycarbophil, dextran, xanthan gum, polyacrylamide, polyacrylamide, crosslinked polymethyl vinyl ether-co-maleic anhydride, commercially available as Gentrez™, gelatin, corn starch and mixtures thereof.

Examples of hydrophobic polymers suitable for formation of the foam are, among others, polypropylene oxide, polyamides, polystyrene, and polymethacrylic acid.

Tampon-like vaginal foams that undergo dissolving or degradation in the vagina into smaller units or polymers by various mechanisms are classified as degradable or dissolving foam. This type of the foam is preferred as long as their degradation or dissolving is controlled and coincides with or exceeds the time needed for a complete release of the drug from the coating attached to the degradable or dissolving vaginal foam.

Non-degradable or non-dissolving vaginal foams are the foams resisting a degradation of the three-dimensional structure. Representative but not limiting examples of non-biodegradable or non-dissolving polymers that may be used exclusively, or in alternative that may be also coated with biodegradable or dissolving polymeric foams, include polyamides, polyethylene, polypropylene, polystyrene, polyvinyl chloride, polymethacrylic acid, and derivatives thereof alone or as co-polymeric mixtures thereof.

Both dissolving or non-dissolving, degradable or non-degradable foams may be prepared in a range of sizes and a variety of shapes suitable for use as a vaginal device or the coating thereof, including foam pillows, tubes, cylinders, spheres, tablets or rings (devices) or films, sheets or beads or any other desirable shape (coating) using an appropriate processes known in the art that introduce porosity in a polymer matrix.

The foam as a vaginal device is preformed into a device such as a tampon, tampon-like cylinder, strip, pad, pillow, tube, sphere, tablet or ring or any other shape as might be desirable or it may be applied as a film, sheet or beads, as a coating to a surface of a more complex vaginal device made of a different material, such as, for example, a conventional vaginal tampon, tampon-like device, pessary, ring, strip, pad, pillow, sheet, tube, sphere or tablet covered by said coating foam. In this configuration the foam is applied as a receptacle for the mucoadhesive composition as described in greater detail in the coating section below.

3. Vaginal Sponge

Another example of the tampon-like device is the vaginal sponge. The mucosal composition comprising a desired therapeutical or health-enhancing agent can be incorporated into a silicone matrix which is coated onto a cylindrical drug-free polyurethane vaginal sponge.

4. Vaginal Ring

Another example of a vaginal device is the vaginal ring. Vaginal rings usually consist of an inert elastomer ring coated by another layer of elastomer containing the drug to be delivered. The rings can be easily inserted, left in place for the desired period of time, up to 7 days, then removed by the user. The ring may be solid or hollow containing the therapeutical and/or health-enhancing agent and it may be coated with an active layer material releasing the drug therefrom. The ring can optionally include a third, outer, rate-controlling elastomer inactive layer coating which contains no drug. Optionally, the third ring can also contain a second drug for a dual release ring. The drug can be incorporated into polyethylene glycol throughout the silicone elastomer ring to act as a reservoir for drug to be delivered.

5. Other Vaginal Devices

Vaginal pessaries, vaginal cylinders, vaginal tablets, vaginal capsules, vaginal pellets, vaginal pads, vaginal patches, vaginal suppositories or vaginal tubes are other examples of drug delivery systems which can be used in the present invention. These systems have been previously used for delivery of vaginal contraceptives, and have been described extensively in the literature.

These other types of vaginal devices are similarly coated on the side or on the end facing the uterus with the coating. For example the pessary or ring can be coated on the side facing the uterus with the other side remaining non-coated, sponge or pad may be coated at the portion closest to the uterus while the other side may be porous and adsorbent for, for example, the menstrual blood.

The vaginal device is provided in dry or wet form or may be wetted prior to insertion.

6. Detailed Description of Figures

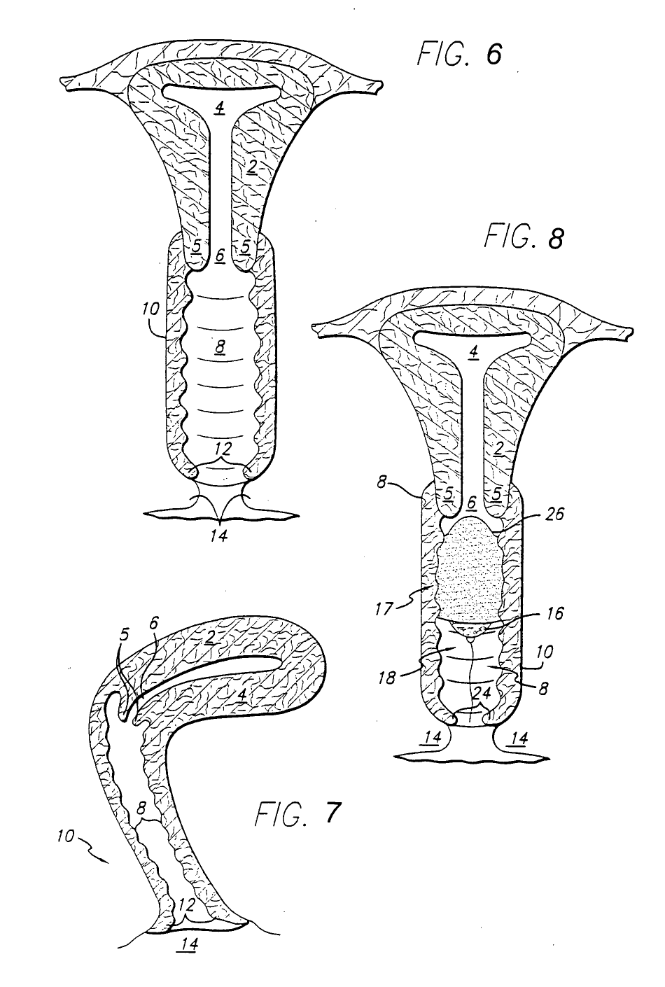

Various embodiments of the vaginal device of the invention vis-a-vis the female reproductive system are illustrated in FIGS. 6-18 using a vaginal tampon or foam as a non-limiting example.

FIG. 6 is a cross-sectional representation of a portion of the female reproductive organs including the uterus and the vagina in the upright orientation. FIG. 7 is a cross-sectional side view representation thereof. The uterus 2 is a muscular organ enclosing the womb 4, and opening at the cervix 5 via the cervical canal or cervical os 6. The vagina 8 is defined by a muscular tube 10 leading from the labia minora 12 and labia majora 14 to the cervix 5. The local vasculature associated with the walls of the vagina 8 communicate with the uterine muscle vascular and lymphatic systems.

FIG. 8 shows placement of a vaginal device 16 in the vagina 8. The vaginal device, represented by a vaginal tampon, is coated in its upper proximal portion with a film coating 17 incorporated with a mucoadhesive composition. Non-coated porous portion 18 of the tampon is seen at the distal end of the tampon. Due to its mucoadhesive properties, the drug or the composition is released from the coating into vagina, adheres to the vaginal mucosa and is transported to the uterus by way of the vaginal blood vascular and lymphatic systems. Physiologically, this concept has been documented and confirmed in animal experiments reported, for example, in the patent U.S. Pat. No. 6,086,909, hereby incorporated by reference.

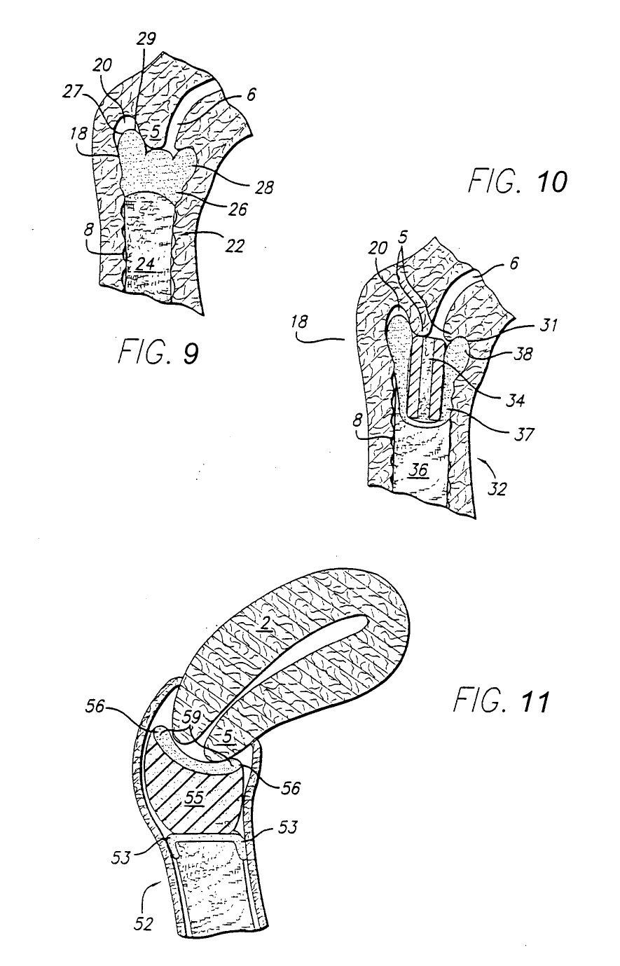

FIGS. 9-14 depict various embodiments of vaginal devices which can be used to deliver the an therapeutical or health-enhancing agent to the uterus or to the general circulation for treatment of migraine and nausea according to the invention.

FIG. 9 is a cross-sectional representation of the vaginal area, adjacent the cervix 5, with a layer attached to the tampon device according to the invention. The tampon device 22 comprises an absorbent cylindrical tampon comprised of non-coated fibrous material 24, for example cotton, at a distal end as well as a coated portion at its proximal end 26 with an annularly positioned a layer in a shape of a cup 29 comprising a composition 28 incorporated into said cup. The proximal end of the tampon device 27 is placed against the upper epithelium 18 of the vagina 8 and posterior fornix 20 for drug delivery through the vaginal mucosa with which the composition 28 is in contact. The composition 28 can be incorporated into a layer coating material, but may also be a powder, melted suppository, foam, paste, or gel composed of suitable delivery components emplaced into the cavity inside the cup.

FIG. 10 is a cross-sectional representation of the vaginal area adjacent the cervix 5 with a tampon device described in the FIG. 9. As seen in the FIG. 10, in the cross section, the cup 31 may contain the mucoadhesive composition 37 incorporated into the cup's wall or such composition may be inserted or placed inside of the cup's inner cavity as a capsule, powder, gel, cream or any other suitable configuration. The cup 31 is pushed against and is in a close proximity to the vaginal mucosa wherein the drug is released. In the shown embodiment, tampon device 32 includes a non-porous tube 34 which communicates with the cervical os 6 for delivery of the a therapeutical or health-enhancing agent from, for example, capsule inserted into the cup inner cavity. Such tube is, of course, an optional feature in this embodiment.

FIG. 11 is a cross-sectional representation of the vaginal area adjacent the cervix 5 which shows an alternate placement of the tampon-like foam device forming a cap coated with a coating wherein the composition is incorporated into the porous foam cap 55 impermeably separated from the body of the tampon with the layer 53. The entire cap 55 or only an upper proximal portion 56 thereof may be incorporated with the mucoadhesive composition or coated with the composition of the invention.

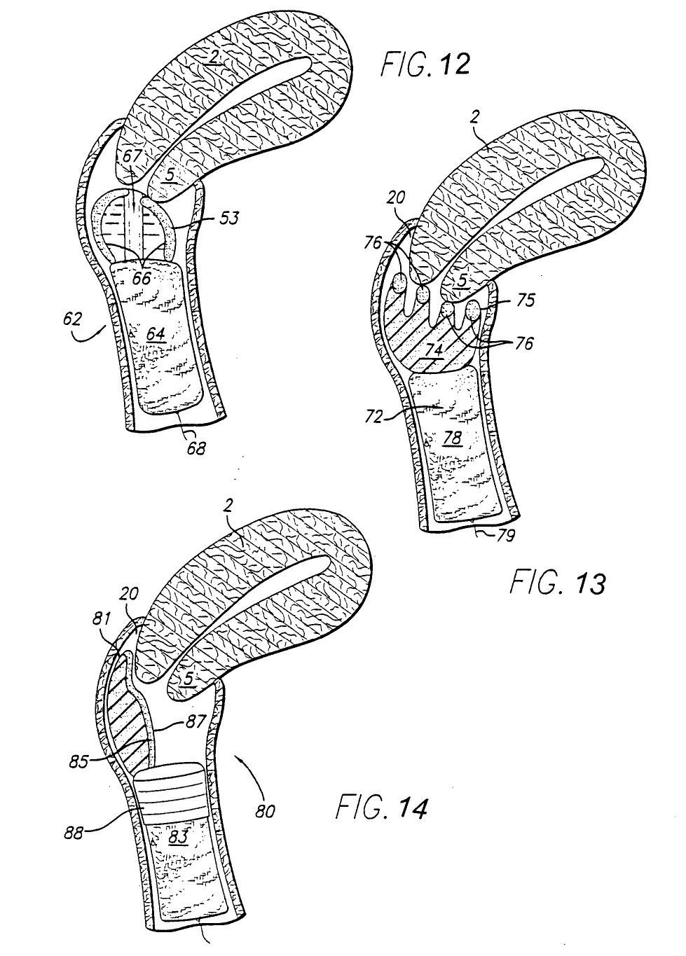

FIG. 12 is a cross-sectional representation of the vaginal area adjacent the cervix 5 showing a tampon 62 coated with a coating 53 with the composition, such as a tablet, capsule, a dissolvable suppository or gel capsule 67 placed within the coating or incorporated into the coating immediately adjacent to the vagina and being available for release to the vagina.

FIG. 13 is a cross-sectional representation of the vaginal area adjacent the cervix 5 with the tampon device 72 including a cap having a protruding fingers 76 which extend into the fornix areas 20 around the cervix 5. The tips of the fingers 76 contain high concentrations of the mucoadhesive composition 75 which may be delivered to more remote areas of vaginal surface.

FIG. 14 illustrates a tampon device 80 having attached thereto a removable cap 88 incorporating a scoop-shaped gel capsule 85 comprising a pharmaceutical composition of the invention. The scoop-shaped porous foam section 85 is annular in shape, but does not completely encircle the cervix 5. Instead, the scoop-shaped porous foam section has a nib-shaped tip 81 which is designed to wedge itself into the posterior fornix 20. The scoop-shaped porous foam section 85 is designed to deliver the mucoadhesive composition to the vaginal wall along the entire length of the scoop-shaped porous foam section 87.

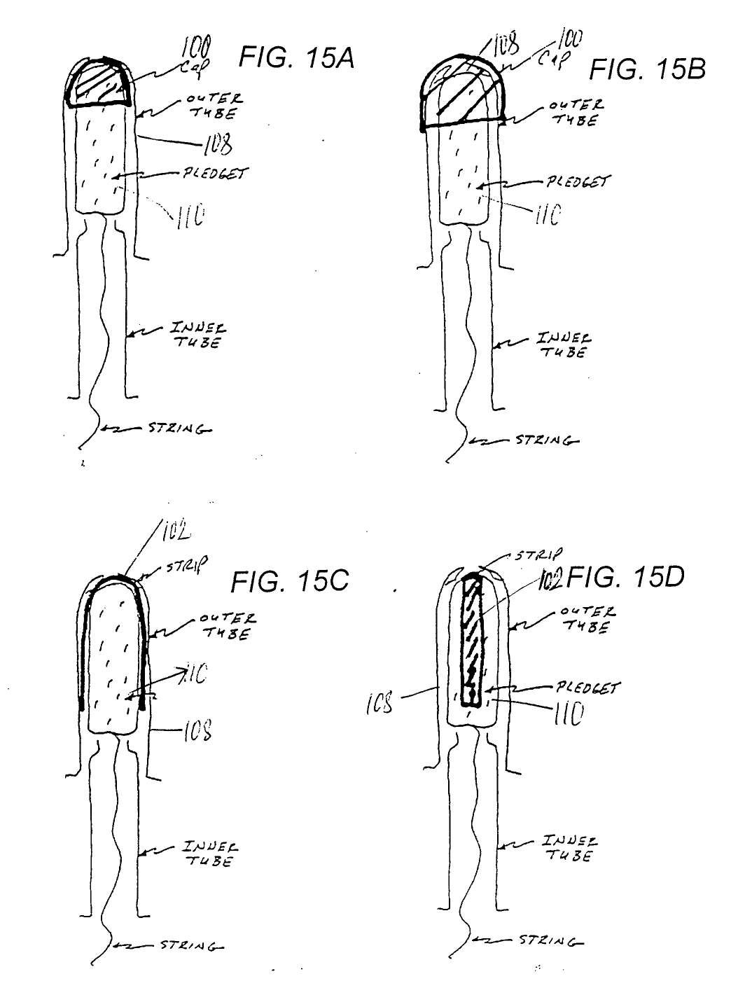

Other alternative arrangement and variations of the vaginal device of the invention are shown in FIGS. 15-20.

FIG. 15 shows a tampon device 110 covered with a cap 100, strip 102 or fiber covering where the cap is positioned over the proximal end of the vaginal tampon (FIG. 15A) or where the coating or covering cap 100 is positioned over the inserting tube 108 of the tampon applicator (FIG. 15B). FIG. 15C shows a side view of the coating or covering strip 102 positioned over the tampon device with a front view of the strip positioned over the tampon seen in FIG. 15D.

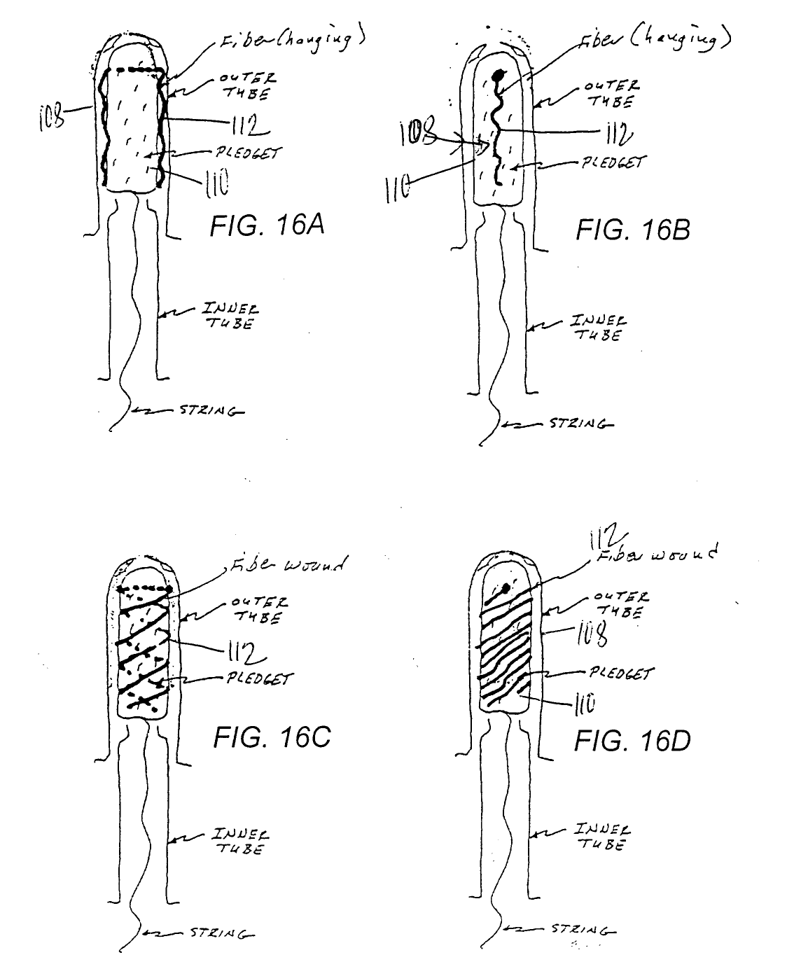

FIG. 16 shows a fiber coating or covering wherein in the fiber 112 is either hanging, inserted through or attached to the tampon (FIG. 16A and FIG. 16B) or wound around the tampon (FIG. 16C and FIG. 16D).

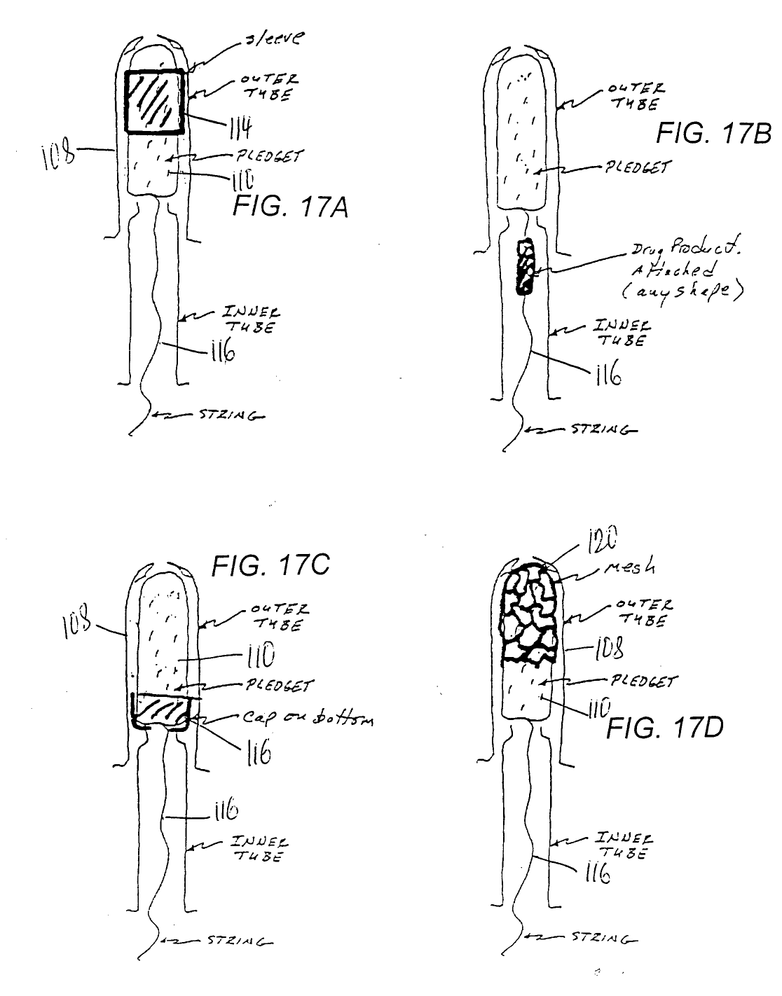

FIG. 17 A shows a strip 114 coating or covering incorporated with a mucoadhesive composition comprising a therapeutic or health-promoting agent applied around the vaginal tampon 110, with an alternative arrangement where the strip is attached to a string 116 attached to the vaginal tampon (FIG. 17B). FIG. 17C shows a coating or covering cap 116 placed around the bottom of the vaginal tampon and the mesh placed as a cap over the top of the tampon 110 seen in FIG. 17D.

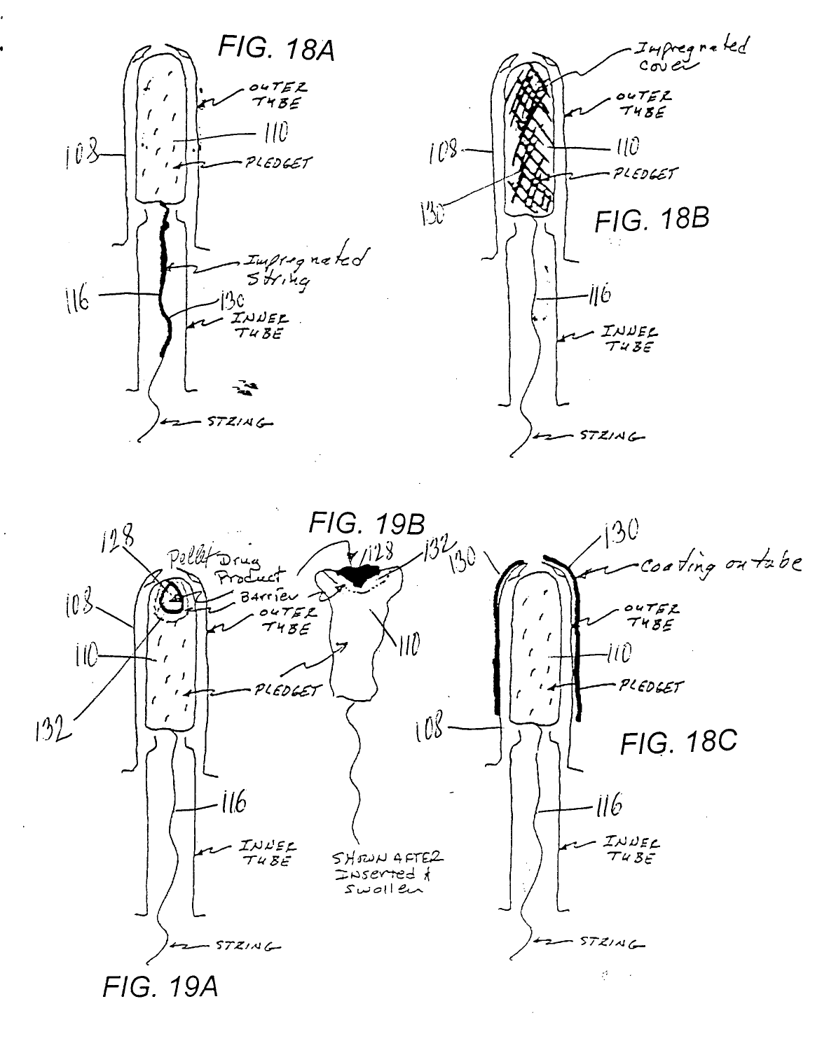

FIG. 18 shows a vaginal device 110 wherein the string 116 attached to the vaginal tampon is impregnated with a coating 130 comprising a mucoadhesive composition comprising a therapeutic or health-promoting agent wherein, seen in FIG. 18B, such coating 130 is applied directly to the tampon. FIG. 18C shows alternative arrangement where the coating is attached to an applicator outer side.

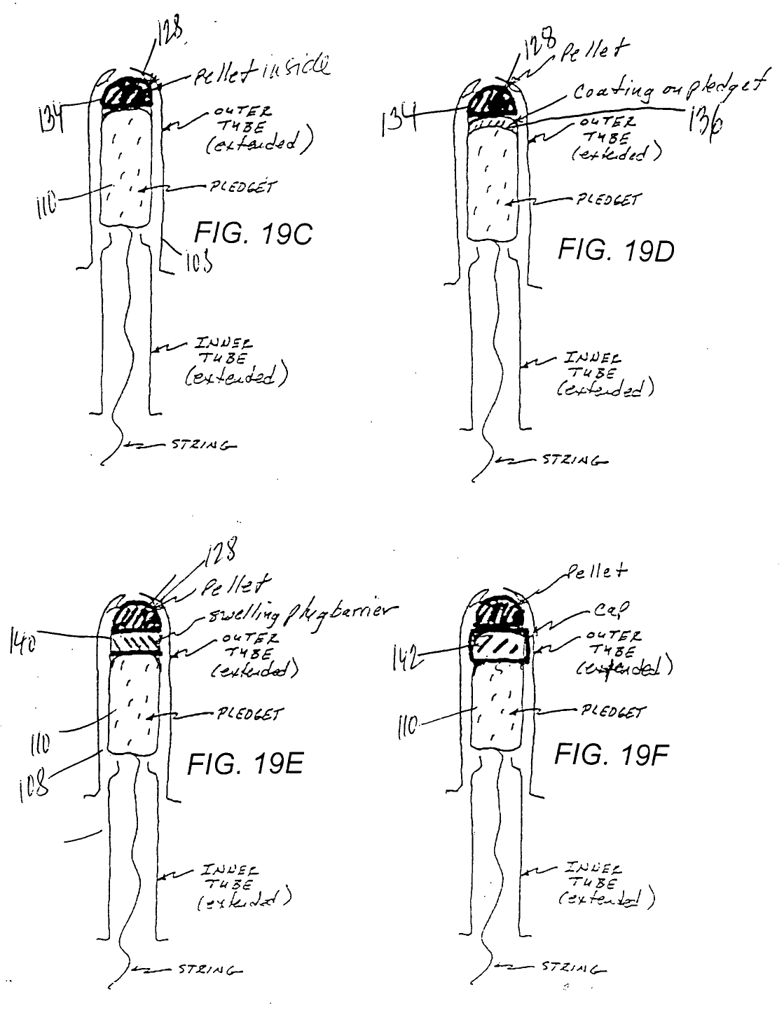

FIG. 19A shows an arrangement where a mucoadhesive composition comprising a therapeutic or health-promoting agent is incorporated into the tablet or pellet 128 sequestered within the tampon 110 body and separated by a coating barrier 132 so that the agent is released exclusively from the pellet or tablet (FIG. 19B). FIG. 19C shows a pellet attached to the tampon withing a coating cap 134. The pellet separated by a coating 136 on the tampon 110 distal end in combination with a covering comprising a pellet 128. The pellet 128 embedded in a swelling plug 140 is seen in FIG. 19E and the pellet 128 separated from the tampon 110 with a swelling cap barrier 142 is shown in FIG. 19F.

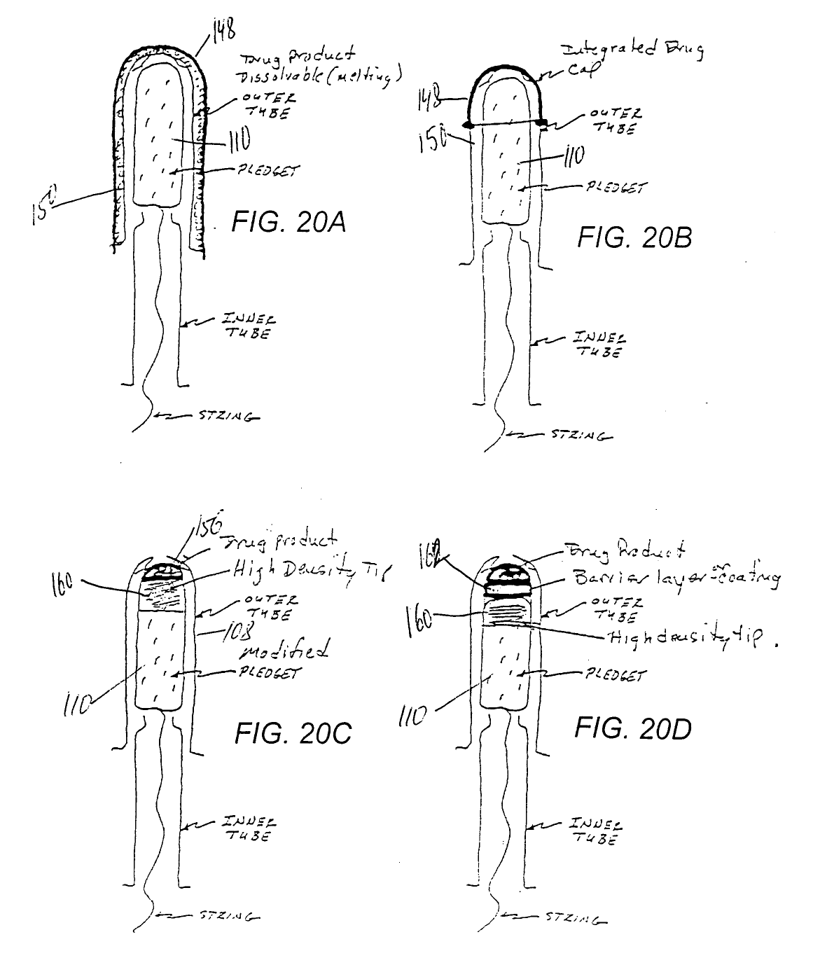

FIG. 20A shows a coating or covering 148 of an applicator tube 150 modified to be made of dissolvable material comprising a mucoadhesive composition comprising a therapeutic or health-promoting agent and its alternative wherein only a portion of the applicator 150 is coated (FIG. 20B). FIG. 20C shows an arrangement wherein a mucoadhesive composition comprising a therapeutic or health-promoting agent is placed in a high density coating 160 on the tip 156 of the vaginal tampon 110 and a variation thereof where there is a barrier 162 coating placed in between the high density coating 160 and between the tampon (FIG. 20D).

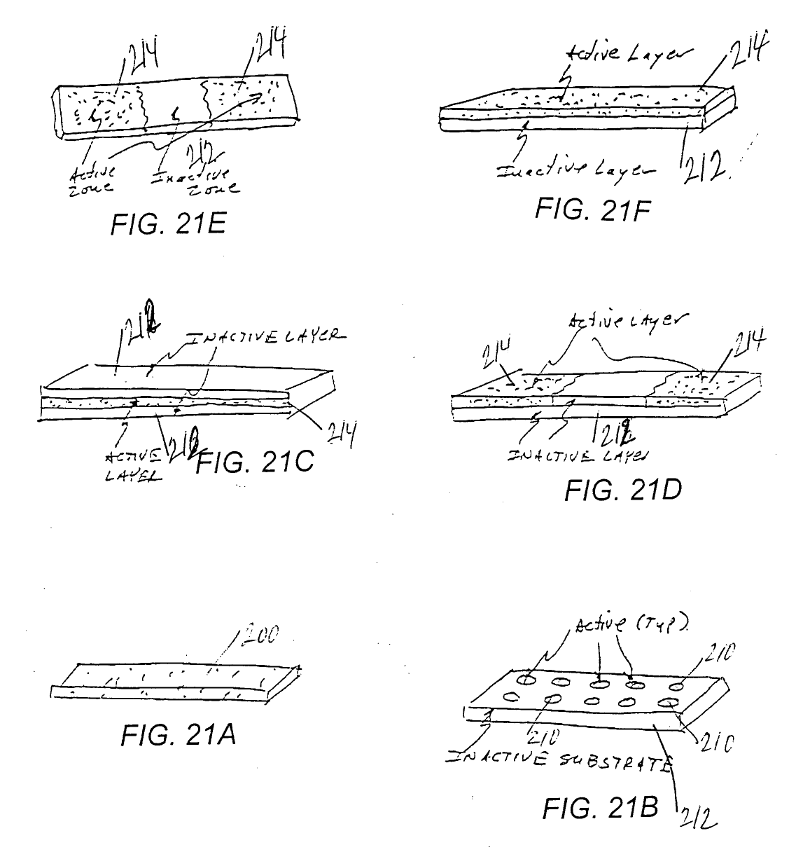

As already discussed above, the coating may be prepared as a single or multiple layer, shaped as a layer covering the device or attached to it as a cap or cup. These features were shown in the above figures. The FIG. 21 illustrates various arrangements within the coating layer or layers showing that the layer may be active, that is it may contain a mucoadhesive composition of the invention or it may be inactive, that is without the mucoadhesive composition. The active layer may be the whole layer or a portion thereof, the active layer may be a portion of the coating layer, may be positioned between two layers or in the middle of one layer. Any and all arrangements are intended to be within the scope of this invention and it is to be understood that the FIG. 21 is presented for illustrative and non-limiting purposes.

FIG. 21 shows exemplary arrangement of the coating or covering according to the invention. A homogeneous layer coating 200 is seen in FIG. 21A, whereas FIG. 21B shows zoned deposits 210 of a mucoadhesive composition comprising a therapeutic or health-promoting agent within an inactive substrate layer 212. FIG. 21C show a three-layer coating with the a mucoadhesive composition comprising a therapeutic or health-promoting agent incorporated into the middle active layer 214 or, as seen in FIG. 21D, active layers 214 are positioned on the sides of the inactive layers 212. Another arrangement, shown in one layer coating is seen in FIG. 21E where the active portions of the coating layer are separated by the inactive layer 212 or where the active layer 214 is placed on top of the inactive layer as seen in FIG. 21 F.

Generally, the tampon device coated with a coating layer, strip, cup or cap is placed in contact with the inner wall of the vaginal mucosa and the therapeutically inactive excipients present in the mucoadhesive composition, namely a lipophilic or hydrophilic carrier, penetration enhancer and mucoadhesive compound act to facilitate the release and adsorption of the drug into the local vasculature. This results in a substantially higher concentration of the drug being delivered to the uterus and/ or to the systemic circulation.

Embodiments of the invention seen in FIGS. 6-21 may include tampon-like devices of a standard length, or may be longer or shorter than standard tampons to facilitate positioning the tampon device closer to or in contact with the vaginal wall or with the cervix, depending on the shape of the device.

Other embodiments of the invention include the device coated or covered with the foam or combined with a foam covering wherein said foam is absorbent and absorbs excess vaginal fluids and thereby provides for improved hygiene or the vaginal device combined with a covering wherein said covering is or includes a suppository or pellet comprising a mucoadhesive composition further comprising a therapeutical agent or health-promoting natural product.

Another embodiment of the invention is the coated vaginal device wherein said coating or covering is a film, foam, strip, cap, cup or particles made of one layer or several layers of polyethylene, low density polyethylene, high density polyethylene, a blend of polyethylene and polypropylene, a copolymer of ethylene-propylene, plasticized polyvinyl chloride, silicone rubber or a combination thereof.

In other embodiment, the invention concerns the coated vaginal device wherein said coating is an attached or detachable cap, cup, film, foam or strip.

In another embodiment of the invention, the vaginal device comprises a mucoadhesive composition comprising a therapeutical agent or a health-promoting natural product, wherein said composition is present in said coating or covering and is formulated for a controlled and sustained time release.

In another embodiment of the invention, the vaginal device is coated with one or several layers wherein a mucoadhesive composition comprising a therapeutical or health-promoting natural agent is incorporated into one active layer of the coating and wherein the second layer of the coating is an inactive layer or wherein said composition is incorporated into an active portion of one or several coating layers, wherein said active portion of the layer may be a middle portion or side portion of the layer.

In another embodiment of the invention, the vaginal device is coated or covered and said coating or covering is formulated to dissolve or disperse in vagina.

In another embodiment of the invention, the vaginal device is coated or covered and said coating or covering is formulated as a lyophilized tablet, foam, film, strip, cap, cup or particles or wherein said coating or covering is formulated as a fast dissolving soft-gel, capsule, tablet, foam, film, strip or foam.

In another embodiment of the invention, the vaginal device for delivering a therapeutical or health-enhancing agent to a female subject wherein said vaginal device is a tampon applicator or applicator-like device including a string attached to said applicator or to a vaginal tampon alone or in combination with a vaginal tampon, vaginal tampon-like device, vaginal ring, vaginal pessary, vaginal foam, vaginal suppository, vaginal pellet or vaginal patch and wherein said vaginal device is partly or completely coated by, covered by, or combined with a covering comprising a film, strip, layer, cap, cup, suppository, pellet, tablet, soft gel, capsule, foam or particles, said coating or covering further comprising the therapeutical or health-enhancing agent.

Based on the above generic description and specific designs seen in FIGS. 6-21, the following designs of the vaginal device are: tampon combined with fil, tampon combined with foam, tampon applicator combined with fil, spray coatings on tampons, soft gel capsule attached to a device as a covering, cap-like cover over the device tip, strip of film placed over the device tip, foam coating over part of the device, fiber helically wound around the device, deformable fiber woven or nonwoven mesh over the device, drug releasing tampon string, drug containing particles associated with a film, foam, spray or capsule.

The drug may be located at the tip (proximal end) of the device, at the bottom (distal end) of the device, on the removal string, on the outer or inner surface of the applicator tube or it may be located on multiple places or have multiple releasing element.

There may be a combination of two or more coatings, coverings or attachment for combining with the device, such as two pellets, one pellet and one coating, swelling barrier, barriers, and any such possible combination of one or several components.

It will be readily apparent to a person skilled in the art that any characterization of the tampon device as having that or another shape is only an approximate description of the vaginal device according to this invention and the device of any shape, form or type that brings the mucoadhesive composition in contact with the adjacent vaginal wall epithelium, and all shapes which conform to the vaginal epithelium and external cervical surfaces are intended to be included within the scope of this invention. Moreover, no term used herein restricts the invention to the use of such devices.

B. Attachment or Incorporation of Mucoadhesive Compositions to the Vaginal Device

The mucoadhesive compositions comprising an therapeutical or health-enhancing agent and their components are described separately in the following section. This section deals only with the attachment or incorporation of these compositions into the layers, foams, films, caps, cups, or strips coating or covering attached to the vaginal device.

The mucoadhesive composition comprising a therapeutical and/or health-enhancing agent is attached to or incorporated into said coating or into a detachable film, foam, cap, cup, suppository, pellet, tablet or strip coverings.

The mucoadhesive composition additionally to the agent also typically comprises a mucoadhesive agent, and/or a lipophilic or hydrophilic carrier and/or a penetration enhancer and/or sorption promoter.

The composition can be a paste, powder, solution, emulsion, cream, or gel having a sufficient thickness to maintain prolonged vaginal epithelium contact. Alternatively, the mucoadhesive composition can be formulated as a coating, a suppository, a sponge, a tablet, a pellet, a capsule, a soft gel or other absorbent material impregnated with a solution, lotion, or suspension of bioadhesive particles, for example. Any form of drug delivery system which will effectively deliver the agent to the vaginal endothelium is intended to be included within the scope of this invention.

Mucoadhesive compositions are either incorporated into, placed on or in, or attached to a coating as a layer, foam, film, cap, cup or strip that act as their delivery vehicles or structures. The agent-containing composition can be incorporated into the coating before or after its attachment to the vaginal device or it can be incorporated only into a portion of said coating by coating the vaginal device or a portion thereof with an active layer containing the mucoadhesive composition of the coating and a portion of the device may be separated from the active layer of the coating by inactive layer not containing said composition or a surface of a prefabricated vaginal tampon or polymeric vaginal foam or other vaginal device may be first coated with inactive layer and then with the active layer. This arrangement separates the absorptive cotton, polymer or foam material of the vaginal tampon from the active layer.

If the vaginal device coated with a coating layer is used, there are numerous methods by which a mucoadhesive composition can be incorporated into the device. For example, the composition can be incorporated into a whole coating layer or into one of the coating layers or a portion thereof, it can be incorporated into the cap covering the tampon, it can be incorporated into a cup surrounding the proximal or distal portion of the tampon, or into a tablet, pellet, soft gel capsule or bioadhesive reservoir placed into or attached to the layer, cap, cup or strip coating or covering at or near the proximate or distal end of the vaginal device.

Alternatively, the drug can be in the form of a liquid or powdered material positioned at the tip of the tampon. The drug can also be absorbed into fibers at the tip of the tampon, for example, by dissolving the agent in a pharmaceutically acceptable carrier and absorbing the agent solution into the fibers. The agent can also be dissolved in a coating material which is applied to the proximate end or the tip of the device and/or placed around the coating attached to the tampon. Alternatively, the agent can be incorporated into an insertable suppository, pellet, soft gel or tablet which is placed in association with the tip of the tampon or is placed in a cup or attached to or on the cap that is either attached permanently to the vaginal device or removable and attached as a covering separately.

One route to deposit the mucoadhesive composition is to spray the device with an active layer coating material containing a mucoadhesive composition in solution or in alternative to spray the device first with an inactive coating and then to spray or add the second active layer containing said composition or attach a covering to the vaginal device wherein said covering is a pre-formed cap, cup, foam, film or strip containing the mucoadhesive composition. Suitable processes to coat the vaginal device are similar to processes used for applying coatings to pills.

Alternatively, the composition can be incorporated into the layer, cup, cap, foam, film or strip by emulsion coating where water-in-oil or oil-in-water emulsions prepared in polymer solution is forced through a prefabricated foam scaffold by applying vacuum. After solvent evaporation, a polymer film containing the agent is incorporated into the structural layer, cap, cup or strip. Processing parameters of this emulsion coating are known to the skilled in the art and any type of process, additives and equipment required to optimize stability and release of the therapeutical and/or health-enhancing agent from within these coatings or coverings are intended to be within the scope of this invention.

C. Coating Layer

The coating material is applied to the device as a layer, layers, cap, cup, particles, foam, film or strip incorporated with the mucoadhesive composition that acts as a continuous agent depot, and depending on the formulation, providing a continuous and uninterrupted delivery of the agent to the vaginal mucosa over a long period of time.

The vaginal device is coated with the coating layer either completely or partially. Preferably the layer coating is applied to the proximal or distal end of the device. Such coating may be a temperature-sensitive material, such as wax, that melts at the body temperature, or one or several layers of degradable or non-degradable thin, supple, non-porous material such as a plastic film, coated gauze, polyethylene, high density polyethylene, synthetic polymers or their combination with polysaccharides such as alginate, dextran, cellulose, collagen or proteins, such as albumin or gelatin, or polyhydroxy acids, such as polylactides, polyglycolides, polyethylene terephthalate, polybutyric acid, polyvaleric acid, polylactide-co-caprolactone, polyanhydrides, polyorthoesters, and blends and co-polymers thereof, or non-degradable polymers such as polyamides, polyethylene, polypropylene, polystyrene, polyvinyl chloride, polymethacrylic acid, and derivatives thereof, or any other suitable coating material that coats at least a portion of the device with a layer, strip or several layers or strips, covers the portion of the device with a cup or cap, or surrounds the device with a coating like a skirt or that opens like an umbrella when it comes in contact with the vaginal environment.

In one embodiment, the coating layer is itself incorporated with the mucoadhesive composition. In other embodiments, the layer is used only as a separating barrier for sequestration of the non-coated part of the vaginal device from the coating or covering structure containing the mucoadhesive composition. In both alternatives, the layer or permanently attached or removable cup, cap or strip or another layer is separated from the non-coated part of the vaginal device. Preferably only the coated part of the device is in contact with the epithelial tissue.

The layer of coating may be applied to the vaginal device as a continuous film, foam, foil or sheet. Additionally, such layer coating may be further coated with another layer of film, foil, foam, sheet, beads, microcapsules, nanocapsules and any such formulation that can conveniently contain the mucoadhesive composition of the invention and release such composition in timely manner when placed in contact with the vaginal mucosa.

Biodegradable polymers suitable for preparation of these films, foils, foams or sheets are preferably designed to allow agent release by bulk or surface erosion and include natural and synthetic polymers alone or in combination with representative but not limiting examples of polysaccharides such as alginate, dextran, cellulose, collagen, and chemical derivatives thereof, proteins such as albumin and gelatin and copolymers and blends thereof, polyhydroxy acids such as polylactides, polyglycolides and co-polymers thereof, polyethylene terephthalate, polybutyric acid, polyvaleric acid, polylactide-co-caprolactone, polyanhydrides, polyorthoesters, and blends and co-polymers thereof.

Physical and chemical properties of coating layers, films, foams, foils or sheets of the invention can be tailored to optimize their intended use, which is achieved by controlling the rate of release of the therapeutical and health-enhancing agent incorporated therein. Agent release from the delivery device can occur by diffusion or erosion, or by a combination of both, leading to immediate and controlled, rapid, slow, continuous or pulsed delivery of the agent to and through the vaginal epithelia.

The rate of agent release depends on physicochemical properties of the agent, on the composition of the film, foil, foam or sheet and also on the surrounding media at the site of administration.

The release of agent from the tampon device should be timed to provide proper uterine concentration of the agent over a typical length of use of a tampon device, usually 1-8 hours. However, when the degradable devices, such as, for example, vaginal foams or sponges are used, the release of the agent can be timed to coincide with he foam degradation time.

The above approach keeps the agent present in the composition from being absorbed into the tampon and thus becoming unavailable for delivery. When the barrier is present the composition absorbs into the vaginal mucosa quantitatively instead of being only partially delivered to the vaginal mucosa and partially absorbed into the tampon. This cap can also be used as a repository for a tablet, gel capsule or the pellet containing the composition of the invention containing the appropriate an therapeutical or health-enhancing agent.

D. Release of the Agent from the Coated Vaginal Device The therapeutic or health-promoting natural agent may be released form the device, from the coating, from the covering or from the covering attached to the device in continuous or pulsating or sustained controlled release manner, depending on the material used for the agent formulation, on the components of the mucoadhesive composition and their ratios, on the manner of their preparation and on the typo of coating or covering used.

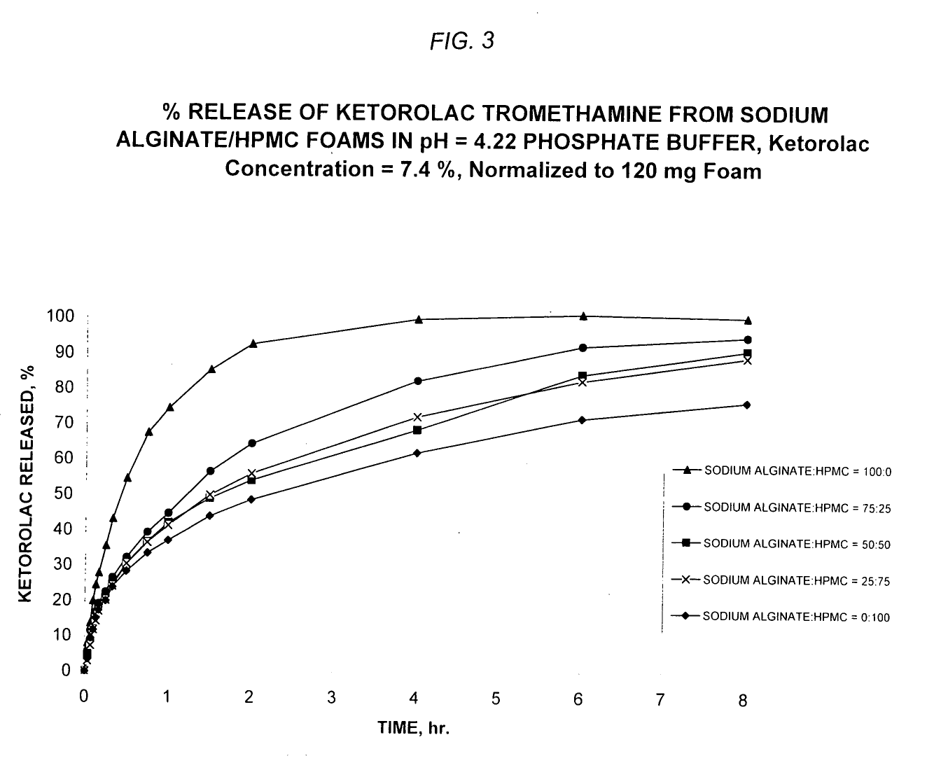

Lyophilized foams prepared according to example 6, for example, provide a controlled release of ketorolac tromethamine, an anti-inflammatory agent, from the lyophioilized foams. FIG. 3 shows a time-release of the drug (%) from the five foam compositions. Results are seen in Table 1 in Example 6.

FIG. 3 shows that the rate of KT release can be varied by modifying the alginic acid (AA): hydropropyl methylcellulose (HPMC) ratio. Using an Erweke DT600 dissolution tester containing 500 ml of pH 4.22 phosphate buffer at 37° C., the fastest initial KT release rate was observed for foams containing only AA polymer; more extended release profiles were observed for AA:HPMC ratios of 75:25 and 25:75. The total amount of KT released from each foam approaches the ideal value of 100% KT released (i.e. 20 mg KT released).

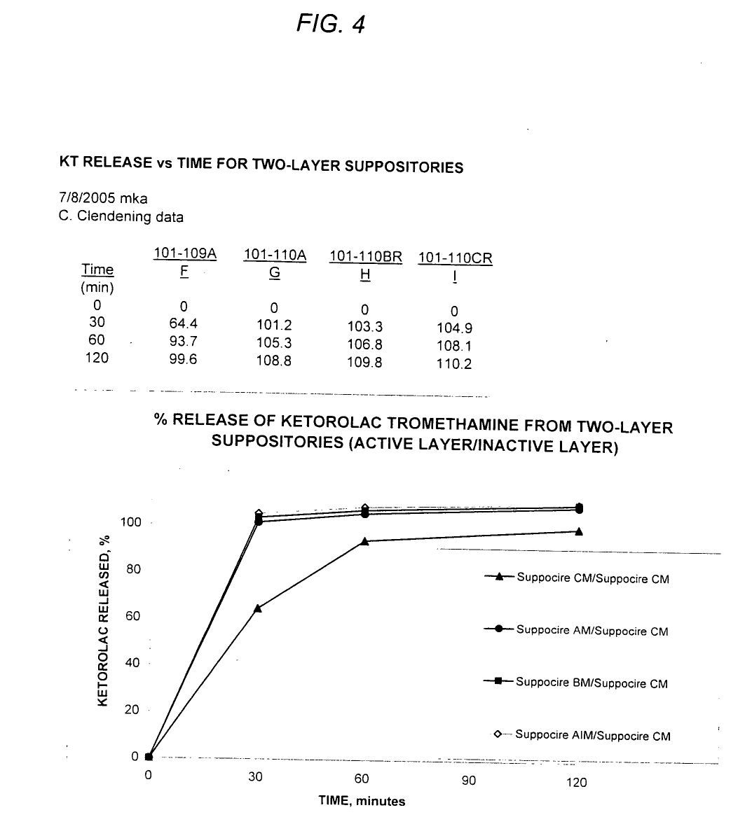

Two layer ketorolac containing suppositories were prepared according to Example 7 and their dissolution was determined. Results are described in Table 2 and shown in FIG. 4.

Dissolution studies were performed using an Erweke DT600 dissolution tester containing 500 ml of phosphate buffer pH 7.0. As shown in FIG. 4, the two-layer pellets released approximately 100% of the available KT within 120 min. Suppositories G, H, and I, advantageously release the 100% of the KT into solution within 30 minutes.

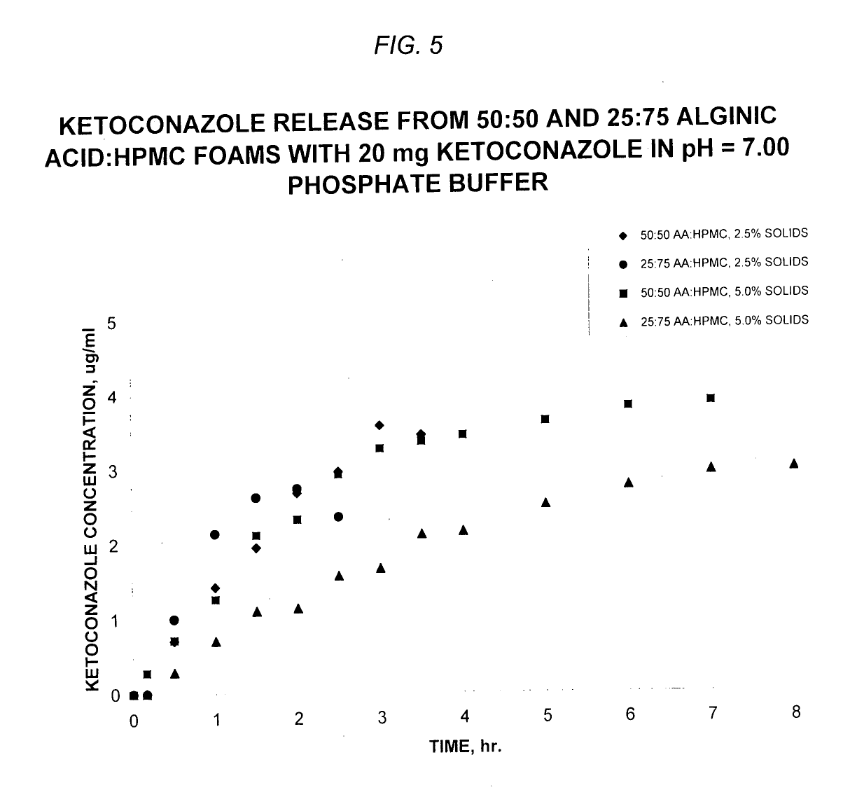

Lyophilized foams prepared according to example 8, for example, provide a controlled release of ketoconazole, an anti-fungal therapeutic agent. Results are summarized in Table 3 and shown in FIG. 5.

FIG. 5 shows a time-release of the drug from the four foam compositions. FIG. 5 shows that the rate of ketoconazole release can be varied by modifying the AA:HPMC ratio as well as the solids content. Using an Erweke DT600 dissolution tester containing 500 ml of pH 7.00 phosphate buffer at 37° C., the fastest initial KT release rate was observed for foams made from a 2.5% solution of 50:50 AA:HPMC. More extended release profiles are obtained from the higher-solids content foams and for AA:HPMC ratios of 25:75.

III. Mucoadhesive Compositions

A mucoadhesive composition of the invention for transmucosal delivery of therapeutical and/or health-enhancing agent consists typically of four essential components. These components are an therapeutical and/or health-enhancing agent that achieves a therapeutically desired effect, a mucoadhesive agent that provides close contact of the composition with the vaginal epithelium, a lipophilic or hydrophilic carrier that assures enhances surface exposure of the agent to the vaginal mucosa, and a permeation enhancer that facilitates transfer of the agent across the vaginal epithelial barrier into the submucosa tissue and systemic blood circulation.

The mucoadhesive composition is typically formulated in therapeutic unit dosage forms and contains a therapeutical and/or health-enhancing agent selected from those already described above. The composition typically contains from 0.00001 to about 45 mg/kg body weight, preferably from 0.001 to 15 mg/kg body weight most preferably 0.1 to 8 mg/kg body weight, of an therapeutical agent and between 0.00001 and 100 mg/kg body weight of a health-enhancing agent, from about 0.1 to about 25% of mucoadhesive agent promoting adhesion of the composition to the vaginal mucosa, from about 5 to about 30% of a permeation enhancer assuring transfer of the agent across the vaginal epithelium, and from about 40 to about 95% of a lipophilic or hydrophilic carrier serving as a vehicle for the agent and, optionally, from about 0 to about 30%, preferably about 1 to 5%, of a solubilizing agent for increased transport of released pharmacological agent into the systemic blood circulation.

Other pharmaceutically acceptable excipients suitable for vaginal delivery, such as buffers, fillers, stabilizers, emulsifiers, and any such other excipients as are known in the art to be useful for these purposes may also be added.

Any component and/or excipient used in formulations of this invention needs to be approved for human use and acceptable for use in the vagina with understanding that not all excipients approved for oral use may be approved and/or suitable for vaginal use.

The mucoadhesive composition is formulated as a solution, gel, cream, lotion, ointment, foam, film, suppository, liposomal suspension, microemulsion, capsule, tablet, microparticles, microcapsules, nanoparticles, or nanocapsules, and each formulation form is either incorporated within a layer, cap or strip of a vaginal device or attached thereto.

The mucoadhesive composition formulated as above can be incorporated into the layer, cap, strip or other coatings or coverings, as described above, of the vaginal device or be used as a coating for such layer, cap or strip. Alternatively, the composition may be incorporated into a sponge, foam, film, tablet, capsule, ring, mucoadhesive patch or iontophoretic system and any one of these may be placed within the cap or attached to the strip or layer.

Any form of agent delivery system that will effectively deliver the therapeutical and/or health-enhancing agent to the vaginal mucosa or transmucosally across the vaginal epithelium into the general blood circulation is intended to be included within the scope of this invention.

A. Components of the Mucoadhesive Composition

Individual components of the mucoadhesive composition are the therapeuticor health-enhancing agent, a mucoadhesive agent, a lipophilic or hydrophilic carrier and penetration enhancer or sorption promoter.

1. Therapeutical and Health-Enhancing Agents

The therapeutical and health- enhancing and promoting agent have been already described above.

The therapeutical or health-enhancing agents are formulated in said composition alone, in admixture of two or more or in admixture of the therapeutical agent and an health-enhancing agent, and/or in combination with another pharmacologically effective agent or with an acceptable pharmaceutical excipient.

2. Mucoadhesive Agents

For vaginal delivery according to the invention, the mucoadhesive composition comprises, as an essential component, a mucoadhesive agent. The mucoadhesive agent permits a close and extended contact of the composition, or the agent released from said composition, with mucosal surface by promoting adherence of said composition or agent to the mucosa. The mucoadhesive agent is preferably a polymeric compound, such as preferably a cellulose derivative, but it may be also a natural gum, alginate, pectin, or such similar polymer. The most preferred cellulose derivative is hydroxypropyl methylcellulose available under the trade name METHOCEL®, commercially available from Dow Chemical Co.

The mucoadhesive agent is present in from about 0.1 to about 25%, by weight, preferably in from about 1.5 to about 15% and most preferably about 1.5-5%.

3. Sorption Promoters

The mucoadhesive composition additionally includes a sorption promoter present in from about 2 to about 30%, by weight. Sorption promoter assures a permeation and penetration of the agent through the vaginal mucosa, that is moving it through the vaginal mucosa and into systemic blood circulation.

Sorption promoters include non-ignitable glycol ester derivatives, such as polyethylene glycol caprylic/capric glycerides known as LABRASOL®, commercially available from Gattefossé, glycol derivatives with glycerol esters, such as oleic acid esters of propylene glycol and glycerol known as ARLACEL® 186, commercially available from Imperial Chemical Industries. Particularly preferred are non-ignitable glycol ether derivatives, such as, most preferably, ethoxydiglycol, known under its trade name TRANSCUTOL® and commercially available from Gattefosse, or interesterified stone oil, for example LABRAFIL M 1944CS, commercially available from Gattefosse. The interesterified stone oil is a vegetable oil ethoxylated by replacing a certain portion of glycerol of the glycerides contained in vegetable oil with polyoxyethylene-glycols.

4. Lipophilic and Hydrophilic Carriers

Depending on the agent affinity, the composition of the invention additionally comprises either the lipophilic or the hydrophilic carrier that is appropriate for the used therapeuticor health-enhancing agent. Such carrier is typically present from about 30 to about 95%, by weight. The carrier is selected from such compounds for which the agent has low affinity. Thus the lipophilic carrier is appropriate and selected for formulation of the hydrophilic therapeutical or health-enhancing agent and the hydrophilic carrier is appropriate for formulation of the lipophilic therapeutical or health-enhancing agent.

i. Lipophilic Carriers

Preferred lipophilic carriers for use with hydrophilic agents include any medium chain triglycerides and/or a saturated mono-, di- or triglyceride of fatty acids, particularly those having carbon chain of from 8 to 18 carbons, or a mixture thereof. Examples of the lipophilic carrier are saturated glycerides known and available under the trade name SUPPOCIRE® AS2 or CS2, and related compounds commercially available, for example, from Gattefosse, Westwood, N.J.

ii. Hydrophilic Carriers

Preferred hydrophilic carriers include polyethylene glycols of molecular weight between about 200 and 8000, OR derivatives or mixtures thereof, such as PEG 6000/PEG 1500, or PEG 6000/PEG 1500/PEG 400, or PEG 6000/PEG 400, or PEG 8000/PEG 1500, commercially available from, for example, Sigma/Aldrich, St. Louis, Mo.

5. Penetration Enhancers

Composition of the invention may additionally contain penetration enhancers, compounds which assist in improving penetration properties of the agent or their mixtures by changing the surface properties of the agents or their mixtures, or agent containing solutions or suspensions. These compounds thus, in a way, act as solubilizers. Examples of the penetration enhancers are non-ionic surfactants. The penetration enhancer may be added from about 1% to about 30%, as required.

6. Solubilizing Agents

The composition optionally includes also a solubilizing agent, such as complex-forming solubilizer citric acid, ethylenediamine-tetraacetate, sodium meta-phosphate, succinic acid, urea, cyclodextrin, polyvinylpyrrolidone, diethylammonium-ortho-benzoate, or micell-forming solubilizers such as Tween and spans, for example Tween 80. Other solubilizer useful for the compositions of this invention are polyoxyethylene sorbitan fatty acid ester, polyoxyethylene-alkyl ethers, n-alkyl amine n-oxides, poloxamers, organic solvents, phospholipids and cyclodextrines.

The solubilizing agents may be added from about 0.1% to about 30%.

7. Additional Excipients

The composition of the invention may additionally contain other excipients, such as, fillers, emulsifiers, stabilizers, buffers, and others, as appropriate. Examples of these excipients are isostearylstearate, isopropyl myristate, glycerin, mineral oil, polycarbophil, carbomer 934P or 940, hydrogenated palm oil, glyceride, sodium hydroxide, sorbic acid, and purified water.

B. Preferred Formulations

All formulations which contains components of the invention in ranges given above are intended to be within the scope of this invention. Few compositions presented here as preferred formulation are only exemplary and are not intended to limit the scope of the invention in any way.

Preferred formulations for a hydrophilic therapeuticor health-enhancing agent comprise between about 0.01-10%, by weight, of the agent, about 60-90%, by weight, lipophilic carrier, between about 0.1-25%, by weight, mucoadhesive agent, between about 1-25%, by weight, sorption promoter and optionally a penetration enhancer or solubilizing agent, usually present in 1-30%, by weight.

Preferred formulations for the lipophilic agents comprise between about 0.01-10%, by weight, of the agent, about 30-90%, by weight of hydrophilic carrier, between about 0.1-25%, by weight, of mucoadhesive agent, between 1 and 25% of sorption promoter and optionally between about 1-30%, by weight, solubilizing agent and/or permeation enhancer.

In one preferred embodiment of the invention, 0.01-10% of the agent is formulated with other components such as between about 60 to 90% by weight lipophilic carrier, between about 1.5 to 20% mucoadhesive agent, between about 10-20% of sorption promoter, between 0 to 30% solubilizing agent, and between about 1 to 30% permeation enhancer.

In another preferred embodiment of the invention, 0.01-10% agent is formulated in admixture with about 60 to 90%, by weight, of hydrophilic carrier, between about 1.5 and about 20% of mucoadhesive agent, between about 10 and 15% of sorption promoter and optionally between 0-30% of solubilizing agent and/or between about 1 and 30% of permeation enhancer.

In another preferred embodiment of the invention, the formulation contains 0.01-10% of a hydrophilic agent, 75% of a lipophilic carrier SUPPOCIRE® AS2, 2% hydroxypropyl methylcellulose, and 15% of ethoxydiglycol (TRANSCUTOL®).

In another preferred embodiment of the invention, the formulation includes 0.01-10% of a lipophilic agent, 75% of a hydrophilic carrier PEG 6000/PEG 1500, 2% hydroxypropyl methylcellulose, and 15% of ethoxydiglycol (TRANSCUTOL®).

C. Process for Formulating Hydrophilic or Lipophilic Therapeutical and Health-Enhancing Agents

The lipophilic or hydrophilic therapeutical or health-enhancing agents are formulated using the following process.

In a general method for preparing a formulation for a hydrophilic agent, the lipophilic carrier is melted at 45-50° C. in a heated vessel. The mucoadhesive agent is added to the carrier with stirring. The preferred hydrophilic agent is dissolved in the sorption promoter combined with the penetration enhancer and solubilizing agent. This mixture is added to the carrier/mucoadhesive agent suspension. The final formulation is poured into molds of the desired size and shape or incorporated into a device of the invention. The molds which are stored in a refrigerator at 4-6° C.

In a general method for preparing a formulation including a lipophilic agent, the hydrophilic carrier is melted in a heated vessel at an appropriate temperature recommended by manufacturer. The mucoadhesive agent is added to the carrier with stirring. The preferred lipophilic agent is dissolved in the sorption promoter, and penetration enhancer combined with the solubilizing agent are optionally added. This mixture is admixed with the carrier/mucoadhesive agent suspension. The final formulation is poured into molds of the desired size and shape or incorporated into a device of the invention. The final formulation is then placed in a refrigerator at 4-6° C.

D. Sustained Release

In one embodiment, the mucoadhesive composition can be formulated as a sustained and controlled release agent system. The therapeutical or health-enhancing agent which is formulated for controlled and sustained release is formulated either for rapid, slow, continuous release or for pulsed delivery.

Continuous release or delivery means continuous and uninterrupted release of the agent from the formulation or device wherein the agent is formulated either in the matrix, microparticle, bioadhesive particle, liposomal suspension or any another system typically used for such release.

Pulsed release or delivery is a delivery of the agent in intermittent intervals. Such pulsed delivery may be provided, for example, by formulating the agent in the matrix, microparticle, bioadhesive particle, liposomal suspension or any another system, as described for continuous delivery, incorporated into individual layers interspaced with inactive layer without agents, such as for example, a layer of dissolvable coatings or by formulating the agent in different formulating agents. Methods and formulating agents for sustained delivery are known in the art.

A delivery system for a controlled release must be capable of controlled release of an therapeutical or health-promoting natural agent into the vaginal mucosa over several hours or more. This is achieved by the addition of time release additives such as hydrogel-forming polymers or non-erodible matrices, etc., known in the art.

Additionally, during the menstrual cycle when the pH of the vagina changes, the agent delivery systems additionally will contain buffers to stabilize pH of the vagina to enhance absorption.

E. Bioadhesive Systems and Microemulsions

Bioadhesive microparticles or bioadhesive nanoparticles constitute still another intravaginal agent delivery system suitable to be incorporated into the layer for use in the present invention.

Bioadhesive systems and microemulsions are formulations particularly suitable for vaginal transmucosal delivery. The microemulsion may contain pharmaceutically acceptable surfactants, for example, LABRASOL®, PLUROL® isostearate (Gattefossé), co-solvents such as isopropanol or ethanol, and water. Microemulsions containing one or more of the above components have been shown to improve bioavailability of therapeuticor health-enhancing agents.

The bioadhesive systems use derivatives of cellulose such as hydroxypropyl cellulose and polyacrylic acid. They release the therapeutical or health-enhancing agents for up to five days once they are placed in the appropriate formulation. This system represents a multi-phase liquid or semi-solid preparation which is easily incorporated into the layers, caps or strips. The microparticles or nanoparticles cling to the wall of the vagina and release the agent over a several hour period of time. Many of these systems were designed for nasal use, as described in U.S. Pat. No. 4,756,907, and 6,200,590 incorporated herein by reference, but can be easily modified for use in the vagina. The bioadhesive system may comprise microparticles or nanoparticles filled with the therapeutical or health-enhancing agent and may contain a surfactant for enhancing solubility and/or uptake of the agent. The microparticles have a diameter of 1-100 μm, whereas nanoparticles have a diameter of 10-1000 nm. Microparticles and nanoparticles can be prepared from starch, gelatin, albumin, collagen, or dextran according to methods known in the art.

All formulating options discussed above may be advantageously incorporated into the coating or covering layers, caps or strips of the vaginal device as described herein.

Bioadhesive tablets are another agent delivery system suitable for transmucosal delivery. These bioadhesive systems use hydroxypropyl cellulose and polyacrylic acid. They release agents for up to five days once they are placed in the appropriate formulation. The tablet of the invention has the shape of a suppository or a tampon so that the maximum contact is achieved between the vaginal wall and the tablet surface or such a shape as is suitable for incorporation into the vaginal device of the invention.

The therapeutical or health-promoting agent formulated in a bioadhesive system may also be incorporated into creams, lotions, foams, paste, ointments, microemulsions, liposomal suspensions, and gels which can be incorporated into the vaginal device. Processes for preparing pharmaceuticals in these vehicles can be found throughout the literature.

Suitable nontoxic pharmaceutically acceptable excipients for use in the compositions of the present invention will be apparent to those skilled in the art of pharmaceutical formulations and examples are described in REMINGTON: The Science and Practice of Pharmacy, 20^{th} Edition, A. R. Gennaro, ed., (2000). The choice of suitable carriers will depend on the exact nature of the particular vaginal dosage form desired, e.g., whether the therapeutical and/or health-enhancing agent is to be formulated into a cream, lotion, foam, ointment, paste, solution, microemulsions, liposomal suspension, microparticles, nanoparticles or gel, as well as on the physicochemical properties of the therapeuticor health-enhancing agent.

Although the mucoadhesive compositions described above typically contain only one agent selected from the group of therapeutical agents or health-enhancing agent, such compositions may additionally contain other pharmaceutical agents or a combination thereof, such as, for example, pain killers, antivirals, antipruritics, corticosteroids and other agents which may enhance the therapeutic effect of the primary therapeutical agent or increase benefit of the health-promoting natural agent.

### Utility

The invention is useful for delivery of a therapeutical and/or health-enhancing agent to the uterus or to the systemic circulation of a female subject. The invention provides several previously unrecognized improvements. The newly described approach provides a coated vaginal device that is configured to be placed and remain in a close contact with a vaginal epithelium wherein the device is coated with or is in association with a covering comprising a mucoadhesive composition able to release a therapeutical or health-promoting natural product therefrom. The new approach provides a more efficacious delivery of the an therapeutical or health-enhancing agent to the uterus or to the systemic circulation. The whole amount of agent released from the composition is available for absorption into the vaginal mucosa instead of being only partially delivered to the vaginal mucosa and partially absorbed by the tampon.

In practice, the current delivery system, that is a composition in combination with a vaginal device of the invention, are applied or administered to a female subject in need of a treatment or desiring to improve health. Typically, the treatment is continued for as long as needed to treat the pathophysiological conditions or to improve health.

### EXAMPLE 1

### Preparation of Sumatriptan Vaginal Suppository

This example describes a process for preparation of intravaginal suppositories for use as a vaginal device or for incorporation into a coating of the vaginal device wherein the coating may be a cup or cap.

The dose of sumatriptan (Global Trade Alliance, Scottsdale, Ariz.) was 20 mg. Vaginal suppositories were formulated and prepared 24 hours prior to administration. The four basic ingredients for the suppositories were distilled water (15% wt), SUPPOCIRE AS2X (Gattefossé, Westwood, N.J.) (67.5% wt), hydroxypropyl methylcellulose (HPMC) (obtained as METHOCEL® K, HPMC K15M, from Dow Chemical, Midland, Mich.) (1.5% wt), a mucoadhesive agent, and TRANSCUTOL® (Gattefossé) (15% wt), a permeation enhancer.

To make eight suppositories, 10.8 grams of SUPPOCIRE, 240 mg of HPMC, 2.4 grams of TRANSCUTOL, and the calculated dose of the agent were weighed out. SUPPOCIRE was melted in a disposable 100 mL polypropylene beaker suspended in a water bath at 50° C. The solution was stirred until completely melted. HPMC and TRANSCUTOL were then added and mixed. Finally, the agent was added combined with 2.4 grams of distilled water. After sufficient mixing, the warm suppository mass was quickly poured into commercial nickel-plated brass suppository molds available from the Adelphi Group of Company (West Sussex, UK). Suppositories were kept refrigerated until use.

### EXAMPLE 2

### Preparation of Metoclopramide Vaginal Suppository

This example describes the preparation of metoclopramide-containing vaginal suppositories for use as a vaginal device or for incorporation into a coating of the vaginal device wherein the coating may be a cup or cap coating or covering.

Metoclopramide hydrochloride is commercially obtained from ICN Biomedicals, Inc. (Costa Mesa, Calif.). Vaginal suppositories comprising a dose of 50 mg per suppository were prepared using the method identical to the procedure described for sumatriptan suppositories. The composition of the pharmaceutical excipients in these formulations was SUPPOCIRE AS2X (66% wt), HPMC (1.5% wt), TRANSCUTOL (15% wt), and distilled water (15% wt).

Suppositories comprising other therapeuticor health-enhancing agents are prepared the same way except that their amount, including of excipients, may vary.

### EXAMPLE 3

### Preparation of Diclofenac Sodium Vaginal Suppository

This example describes the procedure for preparation of hydrophilic diclofenac vaginal suppositories for use as a vaginal device or for incorporation into a coating of the vaginal device wherein the coating may be a cup, cap or an inactive layer coating.

A binary mixture of 7.18 grams of polyethylene glycol (PEG) 3350 and 3.86 grams of PEG 6000 (Fisher Scientific, Pittsburgh, Pa.) is melted on a water bath. To the homogenous PEG solution 400 mg of triethanolamine (Sigma/Aldrich, St. Louis, Mo.) is added. In a separate container, 400 mg diclofenac sodium (Spectrum Chemicals & Laboratory Products, Gardena, Calif.) is dissolved in 2.4 grams of TRANSCUTOL that is further diluted with 2.4 grams of distilled water. Both solutions are combined and cooled under stirring. After reaching suitable viscosity, aliquots of the suppository mass are filled into nickel-plated brass molds.

### EXAMPLE 4

### Preparation of Promethazine Vaginal Film

This example describes the process for preparation of vaginal film composition for use as a coating for a vaginal device or for incorporation into a coating of the vaginal device wherein the coating may be a cup, cap, strip coating or covering.

In a 100 mg glass beaker, 240 mg promethazine hydrochloride (Spectrum Chemicals & Laboratory Products, Gardena, Calif.) is dissolved in 2 grams of distilled water and 1.5 grams of TRANSCUTOL. This agent solution is combined with a polymeric alginic acid solution consisting of 500 mg alginic acid, sodium salt (CarboMer, Inc., Westborough, Mass.) and 8 grams of water. Thin films of approximately 1 mm in thickness will be prepared using a hand-operated CAMAG TLC plate coater (CAMAG Scientific, Inc., Wilmington, N.C.).

### EXAMPLE 5

### Preparation of Metoclopramide Vaginal Foam

This example describes the preparation of a medicated vaginal foam for use as a vaginal device or as a coating for a vaginal device or for incorporation into a coating of the vaginal device wherein the coating may be a cup, cap, or strip coating or covering.

Metoclopramide hydrochloride (ICN Biomedicals, Inc., Costa Mesa, Calif.) is dissolved in a mixture of PEG 400 (10% wt, Fisher Scientific, Pittsburgh, Pa.) and TRANSCUTOL (15% wt). In a separate container 4.5% (wt) alginic acid, sodium salt is dissolved in distilled water (70% wt). Both solutions are combined and aliquots of 5 mL filled into plastic syringe. Following a thorough freezing process at −80° C., the samples were removed from the syringe mold and lyophilized to form the medicated vaginal foam.

### EXAMPLE 6

### Lyophilized Foams for Controlled Release of Ketorolac Tromethamine

This example describes procedures used for preparation of lyophilized foams for controlled release of ketorolac tromethamine.

Lyophilized foams A through E, capable of releasing ketorolac tromethamine (KT) in a controlled manner, were formulated according to Table 1 which summarizes the solution compositions and the estimated compositions of the resulting freeze-dried foams. The formulations were designed such that a 30 mm length of freeze-dried foam contains a 8-9 mg dose of KT. First, HPMC powder (hydroxypropyl methylcellulose) (Dow Chemical) was dispersed with stirring into deionized water heated to 70° C.-85° C.

The ketorolac tromethamine USP micronized (Quimica Sintetica) was next dispersed into the mixture, followed by the sodium alginate (AA) (Aldrich). The hazy mixture was transferred into 10 ml syringes and allowed to cool before freezing the compositions at −80° C. for at least 12 hr. The frozen cylindrical compositions were transferred onto metal trays pre-cooled to −40° C. and freeze-dried for at least 72 hr at approximately −20° C. The KT-containing foams, each weighing about 100-20 mg per 30 mm length, were stored in plastic bags in a desiccant cabinet.

TABLE 1
KETOROLAC TROMETHAMINE RELEASE FROM SODIUM
ALGINATE + HPMC LYOPHILIZED FOAMS

| | A | B | C | D | E |
|---|---|---|---|---|---|
| Solution Formulations | | | | | |
| Sodium Alginate (AA), g | 2.5023 | 1.8779 | 1.2503 | 0.6236 | 0 |
| HPMC, g | 0.0000 | 0.6245 | 1.2507 | 1.8773 | 2.4963 |
| Ketorolac Tromethamine, g | 0.2002 | 0.2022 | 0.2015 | 0.2014 | 0.1999 |
| Deionized Water, g | 100.0 | 100.0 | 100.0 | 100.0 | 100.0 |
| % Dry Weight in Foam | | | | | |
| Sodium Alginate (AA) | 92.59 | 69.4 | 46.3 | 23.1 | 0 |
| HPMC | 0 | 23.1 | 46.3 | 69.5 | 92.59 |
| Ketorolac Tromethamine | 7.41 | 7.48 | 7.46 | 7.45 | 7.41 |
| AA:HPMC Ratio | 100:0 | 75.2:25 | 50:50 | 24.9:75 | 0:100 |
| Foam Length, mm | 30 | 30 | 30 | 30 | 30 |
| Sample Weight, g | 0.1206 | 0.1163 | 0.1071 | 0.1147 | 0.1144 |
| Ketorolac in Sample, mg | 8.936 | 8.699 | 7.99 | 8.545 | 8.477 |
| (estimated from % KT × Sample Weight) | | | | | |

### EXAMPLE 7

### Two-Layer Suppositories Containing Ketorolac

This example describes procedures for preparation of two-layer (active and inactive) suppositories containing ketorolac.

Two-layer suppositories suitable for controlled release of ketorolac tromethamine were made for the compositions listed in Table 2. For the active layer (agent-containing layer) in each suppository formulation, the Suppocire component was melted with gentle heat followed by the addition of KT, Labrafil, and Tween 80. 700 mg of active layer mixture was poured into a mold and allowed to cool. Subsequently about 300 mg of the molten inactive layer composition in Table 2 was poured over the active layers in the molds and allowed to cool, resulting in a two-layer suppository consisting of a KT-containing active layer and a agent-free inactive layer.

TABLE 2
TWO-LAYER SUPPOSITORY FORMULATIONS

| | F | G | H | I |
|---|---|---|---|---|
| % Composition -- | | | | |
| Active Layer | | | | |
| KT | 3.20 | 3.20 | 3.20 | 3.20 |
| Suppocire AM | | 55.25 | | |
| Suppocire BM | | | 55.25 | |
| Suppocire CM | 55.25 | | | |
| Suppocire AIM | | | | 55.25 |
| Labrafil M 1944 CS | 15.98 | 15.98 | 15.98 | 15.98 |
| Tween 80 | 25.57 | 25.57 | 25.57 | 25.57 |
| % Composition -- | | | | |
| Inactive Layer | | | | |
| Suppocire CM | 92.75 | 92.75 | 92.75 | 92.75 |
| HPMC | 7.25 | 7.25 | 7.25 | 7.25 |

### EXAMPLE 8

### Lyophilized Foams for Controlled Release of the Antifungal Drug Ketoconazole

This example describes preparation lyophilized foams for controlled release of the antifungal drug ketoconazole.

Lyophilized foams J through M, capable of releasing ketoconazole in a controlled manner, were formulated according to Table 3 which summarizes the solution compositions and the estimated compositions of the resulting freeze-dried foams. The formulations were designed such that a 30-cm length of freeze-dried foam contains a 20 mg dose of ketoconazole.

HPMC powder (hydroxypropyl methylcellulose) (Dow Chemical) was dispersed with stirring into deionized water heated to 70° C.-85° C. The ketoconazole USP micronized (Quimica Sintetica) was next dispersed into the mixture, followed by the sodium alginate (AA) (Aldrich). The hazy mixture was transferred into 10 ml syringes and allowed to cool before freezing the compositions at −80° C. for at least 12 hr. The frozen cylindrical compositions were transferred onto metal trays pre-cooled to −40° C. and freeze-dried for at least 72 hr at approximately −20° C. The ketoconazole-containing foams, weighing about 140 mg per 30 mm length for foams made from 2.5% solutions and weighing about 260 mg per 30 mm length for foams made from 5.0 solutions, were stored in plastic bags in a desiccant cabinet.

TABLE 3
KETOCONAZOLE RELEASE FROM SODIUM
ALGINATE + HPMC LYOPHILIZED FOAMS

| | J | K | L | M |
|---|---|---|---|---|
| Polymer Sol'n | 2.5 | 2.5 | 5 | 5 |
| Conc, % wt | | | | |
| AA:HPMC Ratio | 50.0:50.0 | 24.9:75.0 | 50.0:50.0 | 25.0:75.0 |
| Nominal KTCN mg | 20 | 20 | 20 | 20 |
| per 3 cm | | | | |
| Alginic Acid, g | 1.2498 | 0.6222 | 2.4987 | 1.2498 |
| HPMC, g | 1.2488 | 1.8758 | 2.501 | 3.7478 |
| Ketoconazole, g | 0.4148 | 0.4141 | 0.4142 | 0.4143 |
| Deionized Water, g | 97.5 | 97.5 | 95 | 95 |
| % Dry Weight in | | | | |
| Foam | | | | |
| Polymers | 85.76 | 85.78 | 92.35 | 92.34 |
| Alginic Acid | 42.84 | 21.37 | 46.15 | 23.09 |
| HPMC | 42.86 | 63.38 | 46.2 | 69.25 |
| Ketoconazole | 14.24 | 14.22 | 7.65 | 7.66 |
| Foam Length, cm | 3 | 3 | 2.95 | 2.9 |
| Foam Diameter, mm | 11.0-12.0 | 11.1-11.9 | 13.0-12.7 | 12.3-13.0 |
| Sample Weight, g | 0.1382 | 0.1397 | 0.2582 | 0.2531 |
| Ketoconazole in | 19.68 | 19.86 | 19.75 | 19.39 |
| Foam, mg | | | | |
| (estimated from % | | | | |
| Drug × Sample | | | | |
| Weight) | | | | |

### EXAMPLE 9

### Preparation of a Suppository-Type Vaginal Pellet Containing Natural Health Product

This example describes procedure used for preparation of a vaginal pellet containing a natural health product containing Vaccinium myrtillus (L.) extract.

1360 mg of a polyethylene glycol having a molecular weight of about 1000 (PEG 1000) and 100 mg PEG 1540 is heated around 60° C. under constant mixing until a clear solution is obtained. 45 mg of standardized Vaccinium myrtillus (L.) extract containing 25% anthocyanosides calculated as anthocyanidins (Organic Herb, Inc., Xinsha, Hunan, China) is added and the stirring continued. The temperature is maintained around 60° C. while 40 mg of anhydrous citric acid under stirring. Following dissolution of the citric acid, 90 mg of Povidone K29-32 is added and the mixture is slowly cooled around 45° C. with stirring. At this point, 45 mg of sodium bicarbonate is added and the suspension stirred for additional 10 min prior to packaging into 12 concave-shaped molds.

### EXAMPLE 10

### Preparation of Lyophilized Foam Containing Natural Health Product

This example describes preparation of the lyophilized foam containing a natural health product.

20 g of Lactobacillus rhamnosus GR-1 culture (1×10⁶ cfu/mL) supplemented with 0.2 μg/mL of p-aminobenzoic acid, 0.0001% D-pantothenic acid, 0.0001% niacinamide, 0.00004% riboflavin and thiamine HCL, 0.0005% L-arginine and L-cystine, 0.005% L-tyrosine, L-tryptophane, and L-aspartic acid is combined with 20 mL of a solution containing 9% alginic acid, sodium salt, 10% PEG 400 in distilled water. Aliquots of 5 mL of the suspension are filled into plastic syringes and subjected to a complete freezing process for 12 h at −80° C. The samples are removed from the syringe mold and lyophilized to yield the probiotic vaginal foam devices.

1. A vaginal device for delivering a therapeutical or health-enhancing agent to a female subject wherein said vaginal device is a vaginal tampon, vaginal tampon-like device, vaginal ring, vaginal pessary, vaginal foam, vaginal suppository, vaginal pellet or vaginal patch; and
wherein said vaginal device is partly or completely coated by, covered by, or combined with, a covering comprising a film, strip, layer, cap, cup, fiber, suppository, pellet, tablet, soft gel, capsule, foam or particles, said coating or covering further comprising the therapeutical or health-enhancing agent.

2. The device of claim 1 wherein therapeutical or health-enhancing agent is formulated as a mucoadhesive composition incorporated into said coating or covering of said device.

3. The device of claim 2 wherein the device is the vaginal tampon or vaginal tampon-like device.

4. The device of claim 2 wherein said mucoadhesive composition comprises at least one therapeutical agent selected from the group consisting of an antimicrobial, vasodilator, nonsteroidal anti-inflammatory (NSAI), prostaglandin inhibitor, COX-1 inhibitor, COX-2 inhibitor, local anesthetic, calcium channel antagonist, potassium channel blocker, β-adrenergic agonist, bisphosphonate, leukotriene blocker, smooth muscle inhibitor, peptide, protein, dyskinetic muscle contraction inhibitor and anti-HIV agent and a combination thereof, or at least one health-promoting agent selected from the group consisting of a botanical, probiotic microorganism, vitamin, antioxidant, anti-pruritic additive and synergistic additive agent and a combination thereof.

5. The device of claim 4 wherein said therapeutical agent is the antimicrobial agent selected from the group consisting of acyclovir, afloxam, amantadine, amphothericin B, azitormycin, bacampicillin, butoconazole, carbenicillin, cefadroxil, cefixime, ceflotoxime, cefpodoxime, cefprozil, cephalexin, cephradine, ciclopirox, ciprofloxacin, clidamycin, clotrimazole, dirithromycin, dosicycline, doxycycline, econazole, erythromycin, famciclovir, fenticonazole, fluconazole, flucytosine, ganciclovir, isoconazole, itraconazole, ketoconazole, lumefloxacin, metronidazole, miconazole, mupirocin, naftifine, norfloxacin, nystatin, oseltamivir, oxiconazole, penciclovir, phosphomycin, ribavirin, rimantidine, sulconazole, terconazole, tetramycin, tioconazole, troleandomycin, voriconazole and zanamivir.

6. The device of claim 4 wherein the therapeutical agent is the non-steroidal anti-inflammatory agent selected from the group consisting of acetaminophen, acetylsalicylic acid, bromfenac, celecoxib, darbufelone, diclofenac, diflunisal, etodolac, etoricoxib, fenamate, fenoprofen, flosulide, flurbiprofen, ibuprofen, indomethacin, ketoprofen, ketorolac, lumiracoxib, meclofenamate, meloxicam, nabumetone, naproxen, nimesulide, oxaprozin, parecoxib, phenylbutozone, piroxicam, rofecoxib, salsalate, sulindac, teroxicam, tolmetin, and valdecoxib.

7. The device of claim 4 wherein the therapeutical agent is the calcium channel antagonist selected from the group consisting of amlodipine, bepridil, diltiazem, felodipine, israpidine, nicardipine, nifedipine, nimodipine, and verapamil.

8. The device of claim 4 wherein the therapeutical agent is the potassium channel blocker selected from the group consisting of 4-aminopyridine, almikalant, ambasilide, amiodarone, apamin, azimilide, charybdotoxin, clofilium, clotrimazole, correolide, dequalinium chloride, dofetilide, glibenclamide, glyburide, ibutilide, paxilline, procain, sematilide, sotalol, tedisamil, tetramethylammonium and tolazamide.

9. The device of claim 4 wherein the therapeutical agent is the beta-adrenergic agonist selected from the group consisting of formoterol, levalbuterol, metaproterenol, pirbuterol, ritodrine, salbutamaol, salmeterol and terbutaline.

10. The device of claim 4 wherein the therapeutical agent is the vasodilator selected from the group consisting of clonidine, dinitrate, doxazosin, guanabenz, guanfacine, hydralazine, isosorbide isosorbide mononitrate, isosorbide dinitrate, methyldopa, minoxidil, nitroglycerin, prazosin, rilmenidine and terazosin

11. The device of claim 4 wherein the therapeutical agent is the bisphosphonate selected from the group consisting of alendronate, alpadronate, clodronate, etidronate, ibandronate, neridronate, olpadronate, pamidronate, residronate, tiludronate and zoledronate.

12. The device of claim 4 wherein the therapeutical agent is the anti-migraine agent selected from the group consisting of ergotamine, dihydroergotamine, ergostine, butalbital, phenobarbital, acetaminophen, diclofenac sodium, ketoprofen, ketorolac, ibuprofen, piroxicam, naproxen, acetylsalicylic acid, flurbiprofen, tolfenamic acid, butorphanol, meperidine, methadone, sumatriptan, naratriptan, razatriptan, zolmitriptan, almotriptan, eletriptan, dexamethasone, hydrocortisone, isometheptene, chlorpromazine, diazepam, droperidol, valproic acid, gabapentin, topiramate and divalproex sodium.

13. The device of claim 4 wherein the therapeutical agent is anti-nausea agent selected from the group consisting of metoclopramide, palonoestrol, gabapentin, olanzapine, doxylamine, prochlorperazine, domperidone, ondansetron, tropisetron, dolasetron, nabilone, dronabinol, levonantradol, aprepitant, cyclizine and promethazine.

14. The device of claim 4 wherein the therapeutical agent is the anti-cancer agent selected from the group consisting of alitretinoid, altretamine, anastrozole, bexarotene, bicalutamide, bisulfan, capecitabine, chlorambucil, cisplatin, docitaxel, doxorubicin, estramustine, etoposide, exemestane, gefitinib, gemcitabine, imatinib, irinotecan, letrozole, lomustine, melphalan, methotrexate, nilutamide, paclitaxel, procarbazine, tamoxifen, temozolomide, thioguanine, topotecan, toremifene, tretinoid and vincristine.

15. The device of claim 4 wherein therapeutical agent is the anti-HIV agent selected from the group consisting of abacavir, amprenavir, atazanavir, delavirdine, didanosine, efavirenz, emtricitabine, enfuvirtide, fosamprenavir, indinavir, lamivudine, lopinavir, nelfinavir, nevirapine, ritonavir, saquinavir, stavudine, tenofovir, zalcitabine, and zidovudine.

16. The device of claim 4 wherein the therapeutical agent is the protein or peptide selected from the group consisting of insulin, parathyroid hormone, calcitonin, vasopressin, oxytocin, interleukin, immunoglobulin A, immunoglobulin G, monoclonal antibodies, oxytocin, humanized antibodies, human growth hormone and a fragment thereof.

17. The device of claim 4 wherein the health-promoting agent is a botanical selected from the group consisting of Agnus castus, aloe vera, comfrey, calendula, dong quai, black cohosh, chamomile, evening primrose, Hypericum perforatum, black currant seed oil, St. John's wort, tea extracts, lemon balm, capsicum, rosemary, Areca catechu, mung bean, borage seed oil, witch hazel, fenugreek, lavender, soy, Vaccinium extract, heath, azaleas, red onion skin, beat root extract, capsanthin and capsaicin.

18. The device of claim 4 wherein the health-promoting agent is a terpenoid selected from the group consisting of 1,8-cineole, agnostinde, aucubin, harpagide, α- and β-pinene, manoalide, oleuropein, vitexin, luteolin 7-O-glucoside, rotundifuran, vitexilactone, casticin, isovitexin, orientin, 6β,7β-diacetoxy-13-hydroxy-λ-8-14-diene, vitexilactone, altissinone, 2″-O-p-hydroxybenzoylorientin, euscaphic acid glucoside ester, γ-linolenic acid, actein, 23-epi-26-deoxyactein and cimiracemoside A.

19. The device of claim 4 wherein the health-promoting agent is a alkaloid selected from the group consisting of arecoline, arecain, guracine, lobeline, papuamine, bastidin, morphine, atropine and vincristine.

20. The device of claim 4 wherein the health-promoting agent is an aliphatic, aromatic, or heteroaromatic organic acid selected from the group consisting of ursolic acid, corosolic acid, epicorosolic acid, maslinic acid, epimaslinic acid, euscaphic acid, gallic acid and caffeic acid.

21. The device of claim 4 wherein the health-promoting agent is a phenol selected from the group consisting of aloin A, aloin B, 7-hydroxyaloin, tannin, gallotannin and menthol.

22. The device of claim 4 wherein the health-promoting agent is a polyketide selected from the group consisting of acemannan, spiramicyn, nystatin, erythromycin, lovastatin, doxorubicin, maytansine and brevetoxin.

23. The device of claim 4 wherein the health-promoting agent is a iridoid selected from the group consisting of agnoside, aucubin.

24. The device of claim 4 wherein the health-promoting agent is a volatile oil, resin or balm selected from the group consisting of aloeresin A and aloeresin B.

25. The device of claim 4 wherein the health-promoting agent is a natural amino acid.

26. The device of claim 4 wherein the health-promoting agent is a mineral selected from the group consisting of calcium, chromium, iron, magnesium, manganese, potassium, selenium and zinc.

27. The device of claim 4 wherein the health-promoting agent is a vitamin selected from the group consisting of riboflavin, thiamine, β-carotene, cyanocobalamine, pyridoxine, ascorbic acid, cholecalciferol and d-α-tocopherole.

28. The device of claim 4 wherein the health-promoting agent is a co-enzyme/factor selected from the group consisting of biotin, choline, folic acid, D-pantothenic acid, lecithin and niacin.

29. The device of claim 4 wherein the health-promoting agent is a probiotic microorganism selected from the group consisting of Lactobacillus acidophilus CRL1259, Lactobacillus brevis CRL1335, Lactobacillus crispatus CTV05, Lactobacillus fermentum RC-14, Lactobacillus rhamnosus GR-1 and Lactobacillus salivarius CRL1328.

30. The device of claim 4 wherein the synergistic additive is selected from the group consisting of caffeine and ethyoxydiglycol.

31. The device of claim 4 wherein the coating comprises at least one pharmaceutically-acceptable excipient selected from the group consisting of a water-soluble polymer, a water-insoluble polymer, a pH buffering agent, surfactant, penetration enhancer, effervescing additive, hydrophilic carrier and lipophilic carrier.

32. The device of claim 31 wherein the excipient is the water-soluble polymer selected from the group consisting of hydroxypropyl methylcellulose, sodium alginate, polyethylene glycol, carbopol, chitosan and propylene glycol alginate.

33. The device of claim 31 wherein the excipient is the water-insoluble polymer selected from the group consisting of microcrystalline cellulose, cellulose fibers, polyethylene and polypropylene.

34. The device of claim 31 wherein the excipient is an inorganic or organic pH buffering agent selected from the group consisting of sodium bicarbonate, sodium carbonate, sodium phosphate, citric acid, sodium citrate, lactic acid, acetic acid, sodium acetate and a combination thereof.

35. The device of claim 31 wherein the excipient is the surfactant selected from the group consisting of Tween 80, sodium lauryl sulfate and a sorbitan ester.

36. The device of claim 31 wherein the excipient is the penetration enhancer selected from the group consisting of ethoxydiglycol, Labrasol, Labrafil, polyyoxymethylene lauryl ether, polyoxyethylene sorbitan monooleate, propylene glycol oleate, polyethylene glycol, bile salt, stone oil and dimethyl sulfoxide.

37. The device of claim 31 wherein the excipient is the effervescing additive selected from the group consisting of citric acid and sodium bicarbonate.

38. The device of claim 31 wherein the excipient is the lipophilic carrier selected from the group consisting of a semi-synthetic glyceride of saturated fatty acids, ethoxylated fatty acid, hard fat and cottonseed oil.

39. The device of claim 4 wherein the mucoadhesive composition comprises at least one component selected from the group consisting of a mucoadhesive agent, water-insoluble additive, surfactant, penetration enhancer.

40. The device of claim 39 wherein the mucoadhesive agent is a water-soluble polymer selected from the group consisting of a cellulose derivative, sodium alginate, pectin, polyvinyl alcohol, polyvinylpyrollidone, polycarbophil and carbopol.

41. The device of claim 40 wherein the cellulose derivative is hydroxypropyl methylcellulose.

42. The device of claim 39 wherein the water-insoluble additive is selected from the group consisting of microcrystalline cellulose, Labrafil, Suppocire AM and hard fat.

43. The device of claim 40 wherein the surfactant is selected from the group consisting of Tween 80, sodium lauryl sulfate and a sorbitan ester.

44. The device of claim 40 wherein the penetration enhancer is selected from the group consisting of ethoxydiglycol, Suppocire AM, Labrafil, polyoxymethylene lauryl ether, polyoxyethylene sorbitan monooleate, propylene glycol oleate, polyethylene glycol, bile salt, stone oil and dimethyl sulfoxide.

45. The device of claim 1 coated or covered with the foam or combined with a foam covering wherein said foam is absorbent and absorbs excess vaginal fluids.

46. The device of claim 1 combined with a covering wherein said covering is a suppository or pellet.

47. The device of claim 4 where the coating is a suppository incorporated with a mucoadhesive composition comprising a therapeutic or health-promoting agent, or a combination thereof.

48. The device of claim 1 wherein said coating or covering is a film, foam, strip, cap, cup or particles comprising one layer or several layers of polyethylene, low density polyethylene, high density polyethylene, a blend of polyethylene and polypropylene, a copolymer of ethylene-propylene, plasticized polyvinyl chloride, silicone rubber or a combination thereof.

49. The device of claim 48 wherein said coating is an attached or detachable cap or cup, film, foam or strip.

50. The device of claim 5 wherein said composition present in said coating or covering is formulated for a controlled and sustained time release.

51. The device of claim 50 wherein said composition is incorporated into one layer of the coating and wherein the second layer of coating is an inactive layer.

52. The device of claim 1 wherein said coating or covering is formulated to dissolve or disperse in vagina.

53. The device of claim 1 wherein said coating or covering is formulated as a lyophilized tablet, foam, film, strip, cap or cup or particles.

54. The device of claim 1 wherein said coating or covering is formulated as a fast dissolving soft-gel, capsule, tablet, film, strip or foam.

55. A vaginal device for delivering a therapeutical or health-enhancing agent to a female subject wherein said vaginal device is a tampon applicator or applicator-like device, alone or in combination with a vaginal tampon, vaginal tampon-like device, vaginal ring, vaginal pessary, vaginal foam, vaginal suppository, vaginal pellet or vaginal patch; and
wherein said vaginal device is partly or completely coated by, covered by, or combined with, a covering comprising a film, strip, layer, cap, cup, suppository, pellet, tablet, soft gel, capsule, foam or particles, said coating or covering further comprising the therapeutical or health-enhancing agent.
